(19) United States
(12) Patent Application Publication
(10) Pub. No.: US 2021/0252047 A1
(43) Pub. Date: Aug. 19, 2021

(54) USE OF HALOGEN COMPOUNDS FOR THE TREATMENT AND PREVENTION OF TISSUE INJURY AND POST-INTENSIVE CARE SYNDROME

(71) Applicants: Faraday Pharmaceuticals, Inc., Seattle, WA (US); Fred Hutchinson Cancer Research Center, Seattle, WA (US)

(72) Inventors: Stephen A. HILL, Seattle, WA (US); Mark B. Roth, Seattle, WA (US); John W. LANGSTON, Seattle, WA (US); Michael L. MORRISON, Seattle, WA (US); Akiko IWATA, Seattle, WA (US); Michael Andrew INSKO, Seattle, WA (US)

(21) Appl. No.: 16/972,718

(22) PCT Filed: Jun. 7, 2019
(86) PCT No.: PCT/US19/36154

§ 371 Date: Dec. 7, 2020

Related U.S. Application Data
(60) Provisional application No. 62/682,574, filed on Jun. 8, 2018.
(60) Provisional application No. 62/730,927, filed on Sep. 13, 2018.
(60) Provisional application No. 62/730,945, filed on Sep. 13, 2018.

(51) Int. Cl. A61K 33/18; A61P 37/06; A61P 9/10

(52) U.S. Cl. CPC A61K 33/18; A61P 9/10; A61P 37/06

(57) ABSTRACT

The present invention relates to the use of halogen compounds, including iodide, to treat and prevent Post-intensive care syndrome and skeletal muscle tissue damage resulting from an injury or disease.

### CROSS-REFERENCE TO RELATED APPLICATIONS

This application claims priority to U.S. provisional application No. 62/682,574, filed Jun. 8, 2018; U.S. provisional application No. 62/730,927, filed Sep. 13, 2018; and U.S. provisional application No. 62/730,945, filed Sep. 13, 2018, all of which are incorporated by reference in their entireties.

### FIELD OF THE INVENTION

This disclosure relates to compositions comprising halogen compounds, including those comprising a halogen compound in a reduced form, e.g. halides, and methods of using halogen compounds, e.g., iodide, for treating or preventing tissue damage following an injury or infection and for treating or preventing post-intensive care syndrome (PICS) and related disorders.

### BACKGROUND OF THE INVENTION

Injuries, illnesses and diseases, and even medical treatments can all result in undesirable secondary injuries or side-effects, which may occur over hours or weeks following the initial insult, i.e., the primary injury. Secondary injuries and side-effects may be caused by a variety of biological processes that occur during or following the primary injury, such as, for example, hemorrhage, edema, ischemia, reperfusion, inflammation, and immune responses. Secondary injuries and side-effects may occur in different regions or locations of a subject, including but not limited to, the brain and nervous system, skeletal muscle tissue, and cardiac muscle tissue. Secondary injuries and side-effects may occur in the same or different regions or locations of a subject than the primary insult. For example, a primary injury to a limb may result in secondary muscle tissue damage in the heart.

Modern advancements in intensive care medicine have improved survival rates of patients with critical illness. However, the associated health care burden imposed by this growing patient population has turned survival of critical illness into a significant medical problem. In parallel with the increase in survival from critical illness and intensive care, the recognition and awareness of particular health and quality of life related problems common among survivors has also grown. Post-intensive care syndrome (PICS) describes a collection of health problems that remains with patients after surviving critical illness and intensive care beyond discharge.

Clinical trials aimed at avoiding intensive care-associated triggers and risk factors have shown some benefit, but there is currently no standard of care or FDA-approved therapeutic intervention for treating or preventing PICS. Given the incidence, persistence, and potential severity of PICS, there exists a need for new treatments for PICS.

Furthermore, primary injuries or diseases that occur within one region of the body may result in damage to tissue located in a different region of the body. Thus, there is a need in the art for composition and methods of reducing the severity of tissue damage that occurs at a site distal from a site of initial disease or injury.

### BRIEF SUMMARY OF THE INVENTION

The present disclosure provides compositions and methods useful in treating or preventing secondary injuries, such as PICS or muscle tissue (e.g., skeletal muscle tissue) damage, that result from a primary injury or disease, and which are located at a site remote from the primary injury or disease, at least in part.

In one aspect, the present disclosure provides a method for treating, reducing the severity of, or preventing PICS in a subject in need thereof, comprising providing to said subject a halogen compound, e.g., iodide, such as NaI. The halogen compound may be present in a composition comprising the halogen compound and a pharmaceutically acceptable carrier, diluent, or excipient, e.g., a pharmaceutical composition. In particular embodiments of any of the methods of the present invention, the PICS or related disorder includes a new or worsening symptom of physical, cognitive, and psychiatric impairment in patients surviving critical illness exhibited during and/or after discharge from intensive care.

In particular embodiments, the disclosure provides methods of treating, reducing the severity of, or preventing any of the cognitive, psychological, or physical impairments of PICS in a subject in need thereof, comprising providing to said subject a halogen compound such as NaI, prior to and/or while receiving intensive care, and/or after receiving intensive care. In particular embodiments, the subject in need thereof may be diagnosed with, exhibit symptoms of, or be otherwise associated with or characterized by, one or more of the following: pain, agitation, confusion, ICU delirium, prolonged length of stay in the ICU, prolonged immobilization, corticosteroid treatment, treatment with neuromuscular blocking agents (NMBAs), malnutrition, old age, acute brain dysfunction, hypoxia, hypoxemia, trauma, hypotension, glucose dysregulation, insulin resistance, hypoglycemia, hyperglycemia, respiratory failure requiring prolonged mechanical ventilation, systemic inflammatory response syndrome (SIRS), severe sepsis, use of renal replacement therapy, acute respiratory distress syndrome (ARDS), prior cognitive impairment, multiple organ dysfunction syndrome (MODS), and multiple organ failure (MOF).

In certain embodiments, the methods disclosed herein are practiced to prevent or reduce the severity or duration of any of the cognitive, psychological, or physical impairments of PICS that may first occur or worsen in a subject during or following receiving intensive care. In particular embodiments, the impairment is selected from one or more of the following: pain, agitation, confusion, ICU delirium, prolonged length of stay in the ICU, prolonged immobilization, corticosteroid treatment, treatment with neuromuscular blocking agents (NMBAs), malnutrition, old age, acute brain dysfunction, hypoxia, hypoxemia, trauma, hypotension, glucose dysregulation, insulin resistance, hypoglycemia, hyperglycemia, respiratory failure requiring prolonged mechanical ventilation, systemic inflammatory response syndrome (SIRS), severe sepsis, use of renal replacement therapy, acute respiratory distress syndrome (ARDS), prior cognitive impairment, multiple organ dysfunction syndrome (MODS), and multiple organ failure (MOF).

In various embodiments of any of the methods of treating, reducing the severity of, or preventing PICS in a subject in need thereof, the cognitive impairment symptoms comprise deficits in executive function, memory, attention, mental processing speed, and problem solving. In various embodiments, the present invention provides methods of treating, reducing the severity of, or preventing one or more cognitive impairment symptoms of PICS, comprising providing to said subject a composition comprising a halogen compound such as NaI, and a pharmaceutically acceptable carrier, diluent, or excipient.

In various embodiments of any of the methods of treating, reducing the severity of, or preventing PICS in a subject in need thereof, the psychological impairment is psychiatric illness in the form of depression, anxiety, or post-traumatic stress disorder. In various embodiments, the present invention provides methods of treating, reducing the severity of, or preventing the psychological impairment symptoms of PICS in a subject in need thereof, comprising providing to said subject a composition of a halogen compound, such as an iodide or NaI, and a pharmaceutically acceptable carrier, diluent, or excipient.

In various embodiments of any of the methods of treating, reducing the severity of, or preventing PICS in a subject in need thereof, the physical impairment is intensive care unit (ICU)-acquired neuromuscular weakness (also referred to herein as ICUAW), which may be diagnosed as, caused by, or manifesting as, critical illness polyneuropathy (CIP), critical illness myopathy (CIM), prolonged neuromuscular blockade, prolonged corticosteroid treatment, mechanical silencing, disuse atrophy, prolonged immobility, poor mobility, frailty, recurrent falls, quadri paresis or tetra paresis. In various embodiments, the methods of the present invention disclose the treatment or prevention of the physical impairment symptoms of PICS in a subject in need thereof, comprising providing to said subject a composition containing a halogen compound, such as an iodide or NaI, and a pharmaceutically acceptable carrier, diluent, or excipient.

In particular embodiments of any of the methods disclosed herein, the methods are practiced to treat or prevent metabolic acidosis, diabetic acidosis, hyperchloremic acidosis, lactic acidosis, or renal tubular acidosis, e.g., metabolic acidosis, diabetic acidosis, hyperchloremic acidosis, lactic acidosis, or renal tubular acidosis, associated with PICS.

In another aspect, the present disclosure provides a method for treating, reducing the severity of, or preventing damage to skeletal muscle tissue resulting from a primary injury or disease in a subject in need thereof, comprising providing to said subject a halogen compound or a pharmaceutical composition comprising a halogen compound and a pharmaceutically acceptable carrier, diluent, or excipient. In certain embodiments, the primary injury or disease is localized to one or more regions of the subject, local tissue damage occurs in one or more of the same regions of the subject as the primary injury or disease, and remote tissue damage occurs in one or more different regions of the subject than the primary injury or disease. For example, the different region may be distal to or at a remote site in the subject as compared to the location of the primary injury or disease. In particular embodiments, a region of a subject is a particular tissue, organ, or limb. In certain embodiments, the secondary injury or remote tissue damage occurs in a different tissue or organ than the primary injury or disease. In certain embodiments, the secondary damage occurs in tissue, e.g., skeletal tissue within one or more limbs, the diaphragm, or torso, of the subject. For example, a patient may experience immediate and localized damage to tissues and organs as a result of blunt trauma to a particular region of the body. Systemic responses to the localized trauma may result in secondary damage to distal regions, such as skeletal muscles unaffected by the initial localized trauma. In certain embodiments, the secondary damage occurs in cardiac tissue of the subject. In certain embodiments, the primary injury or disease is a disease, which results in secondary injury or damage to a localized region of the subject, e.g., tissue damage in muscle tissue, e.g., skeletal muscle, smooth muscle, or cardiac muscle. In some embodiments, the disease results in secondary damage to skeletal tissue within one or more limbs, the diaphragm, or torso, of the subject, or to cardiac tissue of the subject. In certain embodiments, the secondary tissue damage resulting from a primary injury or disease occurs in the diaphragm or intercostal muscle.

The halogen compound may be present in a composition comprising the halogen compound and a pharmaceutically acceptable carrier, diluent, or excipient, e.g., a pharmaceutical composition. In particular embodiments, the halogen compound, e.g., I-, or composition is provided to the subject prior to, during, or following the primary injury or disease. In certain embodiments, the secondary injury is an injury to muscle tissue. In particular embodiments, the muscle tissue is skeletal muscle (including but not limited to limb and respiratory muscles), cardiac muscle tissue or smooth muscle tissue.

In some embodiments of the methods disclosed herein, the primary injury or disease is a localized trauma, e.g., a blunt force trauma, a surgery, a burn injury, an ischemic injury, an ischemia reperfusion injury, a traumatic brain injury, a stroke, or a radiation injury. In some embodiments, the primary injury or disease is an infection, optionally a viral infection, a yeast infection, or a bacterial infection. In some embodiments, the primary injury or disease is a local inflammatory condition, optionally gastritis, pancreatitis, necrotizing enterocolitis, or colitis. In some embodiments, the primary injury or disease has resulted in a systemic inflammatory response syndrome (SIRS) or sepsis in the subject. In some embodiments, the primary injury or disease is sepsis, chronic obstructive pulmonary disease (COPD), chronic or acute heart failure (e.g., left-sided, right-sided, systolic, diastolic or congestive heart failure), uremia, kidney disease, liver disease, cancer, chronic pulmonary disease, cirrhosis, cachexia or any disease in which cachexia is a common feature or symptom. In some embodiments, the primary injury or disease is a disease in which there are frequent acute exacerbations of a chronic condition. In some embodiments, the primary injury or disease is an acute episode or exacerbation of a chronic disease. In some embodiments, the primary injury is caused by a medical treatment or critical care, e.g., chemotherapy or immunotherapy. In certain embodiments of any of the primary injuries or diseases disclosed herein, the secondary injury is muscle dysfunction or weakness, e.g., at a location remote from the primary injury or disease. In particular embodiments, the muscle is smooth muscle, skeletal muscle, or cardiac muscle.

In some embodiments, the method enhances the survivability of the subject following the primary injury or disease. In certain embodiments, the primary injury or disease is present within a different region of the subject than the muscle tissue. In some embodiments, the halogen compound, e.g., I-, or the composition is provided to the subject orally or parenterally. In some embodiments, the halogen compound, e.g., I-, or the composition is provided to the subject as a bolus dose prior to the primary injury or disease, optionally wherein the bolus dose comprises less than or equal to about 10 mg/kg, optionally about 1.0 mg/kg. In some embodiments, the halogen compound, e.g., I-, or the composition is provided to the subject following the primary injury or disease. In particular embodiments, the halogen compound is sodium iodide. In some embodiments, the subject is provided with the halogen compound, e.g., NaI, via repeat daily disease of about 2 mg/kg for several days, e.g., about 3 days, about 4 days, about 5 days, or about 1 week.

In certain embodiments of any of the methods disclosed herein, the halogen compound, e.g., I-, or the composition is provided to the subject in an amount sufficient to increase the blood concentration of the halogen compound at least five-fold, at least ten-fold, at least 50-fold, at least 100-fold, at least 500-fold, at least 1000-fold, at least 10,000-fold, or at least 100,000-fold for at least some time.

In certain embodiments, the composition is a stable liquid pharmaceutical composition comprising the halogen compound, e.g., halide, and one or more pharmaceutically acceptable carriers, diluents, or excipients. In certain embodiments, the composition comprising the halogen compound comprises one or more of a reducing agent, a tonicity agent, a stabilizer, a surfactant, a lycoprotectant, a polyol, an antioxidant, or a preservative. In particular embodiments, the halogen compound is sodium iodide. In particular embodiments, the composition is formulated to maintain the halogen present in the composition in a reduced state, e.g., to maintain iodide in its −1 oxidation state. In certain embodiments of methods and compositions of the present invention, at least 90% of the reduced form of the halogen compound present in the composition remains in a reduced form for at least one hour, at least one week, at least one month, or at least six months when stored at room temperature. In certain embodiments, at least 90% of the reduced form of the halogen compound in the composition is present in a reduced form for at least one month, at least two months, at least four months, at least six months, or at least one year when stored at about 4° C. In particular embodiments, the halogen compound is iodide, e.g., NaI.

In certain embodiments of any of the methods disclosed herein, the halogen present in the halogen compound is in a chemically reduced form, e.g., a halide. In certain embodiments, the halogen compound comprises iodine, bromine, chlorine, fluorine, or astatine. In particular embodiments wherein the halogen compound comprises iodine, said halogen compound is an iodide. In various embodiments, the iodide is sodium iodide, potassium iodide, hydrogen iodide, calcium iodide, silver iodide, lithium iodide, magnesium iodide, or zinc iodide. In certain embodiments, the halogen compound comprises bromine. In particular embodiments wherein the halogen compound comprises bromine, the halogen compound is a bromide.

In various embodiments of methods disclosed herein, the composition is provided to the subject parenterally or orally. In particular embodiments, the composition comprises a stable reduced form of the halogen compound, formulated for intravenous administration, e.g., as a single bolus, or administration by infusion, e.g., continuous infusion over a time period, e.g., 30 minutes to four hours. In certain embodiments, the composition comprises a stable reduced form of the halogen compound formulated for oral administration. In particular embodiments, the halogen compound is iodide, e.g., NaI.

In various embodiments of the methods disclosed herein, the composition is provided to the subject in an amount sufficient to increase the blood concentration of the halogen compound at least 2-fold, at least 5-fold, at least 10-fold, at least 20-fold, at least 30-fold, at least 50-fold, at least 60-fold, at least 70-fold, at least 80-fold, at least 90-fold, at least 100-fold, at least 200-fold, at least 300-fold, at least 500-fold, at least 600-fold, at least 700-fold, at least 800-fold, at least 900-fold, at least 1000-fold, at least 10,000-fold, or at least 100,000-fold for at least some time.

In particular embodiments of any of the methods disclosed herein, the halogen compound contains iodide and is administered such that the concentration of iodide is maintained in the blood at a level of at least 10 nM, 100 nM, 1 uM, 10 uM, 100 uM, 1 mM for at least 1 hour, 12 hours, 1 day, 1 week, or 1 month.

In certain embodiments of any of the methods disclosed herein, the halogen compound contains iodide, and about 10 μg/kg to about 10 g/kg of iodide is provided to the subject. In particular embodiments, about 10 μg/kg to about 10 mg/kg, about 100 μg/kg to about 10 mg/kg, about 0.1 to about 10 mg/kg, about 0.5 to about 10 mg/kg, about 0.5 to about 2 mg/kg, about 1 mg/kg to about 10 mg/kg, about 0.1 mg/kg, about 0.5 mg/kg, about 1 mg/kg to about 5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or about 100 mg/kg, of iodide or sodium iodide is provided to the subject.

In certain embodiments, the halogen compound is provided to the subject prior to a scheduled medical procedure, e.g., a scheduled surgery, or an emergency medical treatment, or within about 1 hour, 2 hours, 4 hours, 8 hours, 12 hours, 24 hours, 48 hours, or 96 hours, following a medical procedure, e.g., a surgery or scheduled or emergency medical treatment. In certain embodiments, any of these amounts are provided to the subject within about 1 hour, 2 hours, 4 hours, 8 hours, 12 hours, 24 hours, 48 hours, or 96 hours, following an injury or trauma, e.g., a surgery.

### BRIEF DESCRIPTION OF THE DRAWINGS

FIGS. 1A and 1B are graphs showing creatine kinase (FIG. 1A) and cardiac troponin (FIG. 1B) levels following ischemia reperfusion injury when animals were treated with the indicated amounts of sodium iodide or sham treatment. Each point represents the result from one animal, and * indicates p<0.05.

FIG. 2 is a graph showing edema formation (wet weight/dry weight) in muscle or lung when treated with iodide or control. The concentration indicated in the legend from top to bottom are shown left to right in the graph for each tissue. Each circle represents the result from one animal, and * indicates p<0.05.

FIG. 3 is a graph showing creatine kinase activity levels following treatment with the indicated amounts of sodium iodide or sham treatment.

FIGS. 4A-4C are graphs showing concentrations of BUN (FIG. 4A), ALT (FIG. 4B), and AST (FIG. 4C) following treatment with the indicated amounts of sodium iodide or sham treatment. Each point represents the result from one animal.

FIG. 5 is a graph showing the survival over time of animals treated with vehicle only (bottom line) or iodide (top line).

FIGS. 6A-6B includes graphs showing mouse HLI survival time following reperfusion over the first 5 days (FIG. 6A) or after 50 days (FIG. 6B) when treated with vehicle only or iodide. In each graph, the top line at the latest time point is 1 mg/kg iodide, and the bottom line at the latest time point is vehicle control.

FIGS. 7A-7D provides graphs showing that cytokine levels in muscle tissue are significantly reduced with NaI administration 24 hours after reperfusion. FIG. 7A shows interleukin-6 (IL-6); FIG. 7B shows interleukin-10 (IL-10); FIG. 7C shows (C—X—C motif) ligand 1 (CXCL1 or KC); and FIG. 7D shows macrophage inflammatory protein 2 (MIP-2.

FIG. 8 provides graphs showing that cytokine levels in plasma are significantly reduced with FDY-5301 administration 24 hours after reperfusion.

FIG. 9 provides a heat map of various cytokines assessed in muscle tissue.

FIG. 10 provides a heat map of various cytokines assessed in plasma.

### DETAILED DESCRIPTION OF THE INVENTION

The present invention includes inter alia methods and compositions related to the use of a halogen compound, e.g., I-, to treat, inhibit, reduce the severity of, or prevent secondary injury or damage to a subject resulting from a different primary injury, disease, disorder, or medical treatment.

In one aspect, the halogen compound (e.g., I-) treats, inhibits, reduces the severity of, or prevents secondary tissue damage resulting from a primary injury, disease or disorder. In particular embodiments, the tissue damage is a direct or indirect result of the primary injury, disease, or disorder. In particular embodiments, the tissue is located at a site distal or remote from the site of the primary injury or disease. In certain embodiments, the secondary tissue damage a period of time passes before the secondary tissue damage occurs. In particular embodiments, the tissue where the damage occurs is muscle tissue, e.g., cardiac muscle tissue, smooth muscle tissue, or skeletal muscle tissue. For example, as shown in the accompanying Examples, damage to a subject's lung tissue and cardiac tissue resulting from ischemia reperfusion injury to hind limbs was reduced by treating the subject with iodide.

In one aspect, the halogen compound (e.g., I-) treats, inhibits, reduces the severity of, or prevents PICS. In particular embodiments, the PICS is a secondary injury resulting from a medical treatment or critical care.

In addition, the present invention includes methods and compositions related to the use of a halogen compound in combination with one or more additional active agents to treat, inhibit, reduce the severity of, or prevent PICS, or to treat, inhibit, reduce the severity of, or prevent of tissue damage resulting from a primary injury, disease or disorder. These methods include providing to a subject a composition comprising a halogen compound in combination with an additional composition comprising the one or more additional active agent, as well as methods that include providing to the subject a single composition comprising both the halogen compound and optionally the one or more additional active agents. The compositions may be formulated for a variety of different routes of administration, including but not limited to, intravenous administration, administration by infusion, or oral administration.

### Definitions and Abbreviations

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

The term “including” is used to mean “including but not limited to.” “Including” and “including but not limited to” are used interchangeably.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) less than an amount or level described herein, for example an amount that is 10%, 20%, 30%, 40%, 50%, 60%, 70%, 80%, or 90% of an amount or level described herein.

A “composition” can comprise an active agent, e.g., a halogen compound; and a carrier, inert or active, e.g., a pharmaceutically acceptable carrier, diluent or excipient. A composition may be a pharmaceutical composition. In particular embodiments, the compositions are sterile, substantially free of endotoxins or non-toxic to recipients at the dosage or concentration employed.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The terms “mammal” and “subject” includes human and non-human mammals, such as, e.g., a human, mouse, rat, rabbit, monkey, cow, hog, sheep, horse, dog, and cat.

“Pharmaceutically acceptable salts” include sulfate, citrate, acetate, oxalate, chloride, bromide, iodide, nitrate, bisulfate, phosphate, acid phosphate, lsomcotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dimtrobenzoate, hydroxybenzoate, methoxybenzoate, methylbenzoate, o-acetoxybenzoate, naphthalene-2-benzoate, isobutyrate, phenylbutyrate, alpha-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, mcotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts. The term “pharmaceutically acceptable salt” also refers to a salt of an antagonist of the present invention having an acidic functional group, such as a carboxylic acid functional group, and a base. Suitable bases include, but are not limited to, hydroxides of alkali metals such as sodium, potassium, and lithium, hydroxides of alkaline earth metal such as calcium and magnesium, hydroxides of other metals, such as aluminum and zinc, ammonia, and organic amines, such as unsubstituted or hydroxy-substituted mono-, di-, or tri-alkylamines, dicyclohexylamine, tributylamine, pyridine, N-methyl, N-ethylamine, diethylamine, triethylamine, mono-, bis-, or tris-(2-OH-lower alkylamines), such as mono-, bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine, N-methyl-D-glucamine, and amino acids such as arginine, lysine, and the like. The term “pharmaceutically acceptable salt” also includes a hydrate of a compound of the invention.

The terms “tissue” and “organ” are used according to their ordinary and plain meanings. Though tissue is composed of cells, it will be understood that the term “tissue” refers to an aggregate of similar cells forming a definite kind of structural material. Moreover, an “organ” pertains to a group of tissues that perform a specific function or group of functions or is a particular type of tissue. In certain embodiments, the tissue or organ is “isolated,” meaning that it is not located within an organism.

The term “buffer” as used herein denotes a pharmaceutically acceptable excipient, which stabilizes the pH of a pharmaceutical preparation. Suitable buffers are well known in the art. Suitable pharmaceutically acceptable buffers include but are not limited to acetate-buffers, histidine-buffers, citrate-buffers, succinate-buffers, tris-buffers and phosphate-buffers. In certain embodiments, the concentration of the buffer is from about 0.01 mM to about 1000 mM, about 0.1 mM to about 1000 mM, about 0.1 mM to about 500 mM, about 0.1 to about 200 mM, about 0.1 to about 100 mM, about 1 mM to about 1000 mM, about 1 mM to about 500 mM, about 1 mM to about 200 mM, about 1 mM to about 100 mM, about 1 mM to about 50 mM, about 2 mM to about 60 mM, about 4 mM to about 60 mM, or about 4 mM to about 40 mM, about 5 mM to about 20 mM, or about 5 mM to about 25 mM.

The term “tonicity agent” or “tonicity modifier” as used herein denotes pharmaceutically acceptable agents used to modulate the tonicity of a composition. Suitable tonicity agents include, but are not limited to, sodium chloride, sorbitol, trehalose, potassium chloride, glycerin and any component from the group of amino acids, sugars, as defined herein as well as combinations thereof. In certain embodiments, tonicity agents may be used in an amount of about 1 mM to about 1000 mM, about 1 mM to about 500 mM, about 5 mM to about 500 mM, about 10 mM to about 450 mM, about 20 mM to about 400 mM, about 50 mM to about 300 mM, about 100 mM to about 200 mM, or about 125 mM to about 175 mM. In certain embodiments, a tonicity agent comprises an amino acid present in a composition at about 5 mM to about 500 mM.

An “antioxidant” refers to a molecule capable of slowing or preventing the oxidation of other molecules. Antioxidants are often reducing agents, chelating agents and oxygen scavengers such as thiols, ascorbic acid or polyphenols. Non-limiting examples of antioxidants include ascorbic acid (AA, E300), thiosulfate, methionine, tocopherols (E306), propyl gallate (PG, E310), tertiary butylhydroquinone (TBHQ), butylated hydroxyanisole (BHA, E320) and butylated hydroxytoluene (BHT, E321).

As used in the specification and appended claims, unless specified to the contrary, the following terms have the meaning indicated:

“Mammal” includes humans and both domestic animals such as laboratory animals and household pets, (e.g., cats, dogs, swine, cattle, sheep, goats, horses, and rabbits), and non-domestic animals such as wildlife and the like.

“Optional” or “optionally” means that the subsequently described event or circumstances may or may not occur, and that the description includes instances where said event or circumstance occurs and instances in which it does not.

“Pharmaceutical composition” refers to a formulation of a compound and a medium generally accepted in the art for the delivery of the biologically active compound to mammals, e.g., humans. Such a medium may include any pharmaceutically acceptable carriers, diluents or excipients therefore.

“Iodide” and “a reduced form of iodide” both refer to iodide, which has a −1 valence state (e.g., NaI). “A reduced form of iodine” includes iodide.

“Therapeutically effective amount” refers to that amount of a compound or composition of the invention that, when administered to a biological material, e.g., a mammal, preferably a human, is sufficient to effect treatment, as defined below, of a disease, injury, or condition in the biological material, e.g., mammal, preferably a human. The amount of a compound or composition of the invention which constitutes a “therapeutically effective amount” will vary depending on the compound or composition, the disease, injury or condition and its severity, the manner of administration, and the age of the biological material, e.g., mammal, to be treated, but can be determined routinely by one of ordinary skill in the art having regard to his own knowledge and to this disclosure.

“Treating” or “treatment” as used herein covers the treatment of the disease, injury, or condition of interest, e.g., PICS or a tissue injury, in a biological material, e.g., mammal, preferably a human, having the disease or condition of interest, and includes: (i) preventing or inhibiting the disease, injury, or condition from occurring in a biological material, e.g., mammal, in particular, when such mammal is predisposed to the condition but has not yet been diagnosed as having it; (ii) reducing the severity or duration of the disease, injury or condition, e.g., when it occurs, e.g., in a mammal predisposed to the condition; (iii) inhibiting the disease, injury, or condition, i.e., arresting its development; (iv) relieving the disease, injury, or condition, i.e., causing regression of the disease or condition; or (v) relieving the symptoms resulting from the disease, injury, or condition. In certain embodiments, as used herein, the term “prevention” includes inhibiting or impeding the onset or progression of a disease or injury, or reducing the amount of injury or damage caused by a disease or injury. As used herein, the terms “disease,” “disorder,” and “condition” may be used interchangeably. As used herein, the term “injury” includes unintentional injuries and intentional injuries, including injuries that occur, “at the hand of man,” including injuries associated with medical procedures, such as surgeries and transplantations.

Halogen Compounds

Certain embodiments of the present invention relate to halogens, which include any element included in Group 17 of the periodic table. Halogen-containing compounds are also referred to as “halogen compounds.” In some embodiments, a halogen compound refers to any compound containing Fluorine, Chlorine, Bromine, Iodine, Astatine, or Ununseptium. In particular embodiments, the halogen-containing compounds are halides, i.e., salts of halogens in the −1 oxidation state. In particular embodiments, the present invention relates to halogen compounds in a reduced form, e.g., iodide. In certain embodiments, other forms of halogen compounds may be used according to the present invention, including, e.g., hydrogen halides, metal halides, interhalogen compounds, organohalogen compounds, and polyhalogenated compounds.

Fluorine (F), the lightest halogen, is the non-metal element with atomic number 9. Under standard pressure and temperature it exists as a diatomic gas F₂. Fluorine is the most chemically reactive element, reacting with all other elements except oxygen, helium, neon, and krypton. It is also the most electronegative element, thus attracting electrons more strongly than all other elements. There are 11 fluorine isotopes with known half-lives, said isotopes having mass numbers ranging from 15 to 25. Natural Fluorine, however, consists of one stable isotope, ¹⁹F.

Chlorine (Cl), the second lightest halogen, is the non-metal element with atomic number 17. Under standard pressure and temperature it exists as a diatomic gas F₂. Chlorine is the element with the highest electron affinity, and the third highest electronegativity. There are 16 chlorine isotopes with known half-lives, said isotopes having mass numbers ranging from 31 to 46. Naturally occurring chlorine is a mixture of two stable isotope ³⁵Cl and ³⁷Cl, existing in natural abundance ratios of approximately 3:1.

Bromine (Br), the third lightest halogen, is the non-metal element with atomic number 35. Under standard pressure and temperature it exists as a diatomic liquid Br₂. There are 26 bromine isotopes with known half-lives, said isotopes having mass numbers ranging from 68 to 94. Naturally occurring bromine is a mixture of two stable isotope ⁷⁹Cl and ⁸¹Cl, existing in natural abundance ratios of approximately 1:1.

Iodine (I), the second heaviest natural halogen, is the non-metal element with atomic number 53. Under standard pressure and temperature it exists as a solid diatomic I₂ molecule. There are 34 iodine isotopes with known half-lives, said isotopes having mass numbers ranging from 108 to 144. Natural iodine, however, consists of one stable isotope, ¹²⁷I.

Astatine, the heaviest natural halogen, is a highly radioactive the non-metal element with atomic number 85. It decays so rapidly (longest half-life less than 12 hours) that its properties are not known with great certainty. It is debated if astatine exists as a diatomic At₂ molecule, as this form has never actually been observed. Astatine can react with hydrogen to form hydrogen astatide, and it is predicted to react with metals such as sodium to form salts. There are 37 known astatine isotopes, all of which are radioactive, with very short half-lives. Said isotopes have mass numbers ranging from 207 to 221. No stable isotopes of astatine exist.

In various embodiments, compositions and methods of the present invention comprise one or more halogen compounds, such as various forms of iodine or bromine.

In one embodiment, the present invention relates to a halogen compound containing iodine. In particular embodiments, the halogen compound contains a reduced form of iodine, such as iodide. Certain embodiments may comprise an iodine-containing halogen compound that is an iodide, iodate, organoiodide, periodate, or periodinane.

In some embodiments, said halogen compound is an iodide comprising one or more compounds from the non-limiting list of Aluminium iodide, Aluminium monoiodide, Ammonium iodide, Antimony triiodide, Arsenic diiodide, Arsenic triiodide, Barium iodide, Beryllium iodide, Bismuth(III) iodide, Boron triiodide, Cadmium iodide, Caesium iodide, Calcium iodide, Candocuronium iodide, Carbon tetraiodide, Cobalt(II) iodide, Coccinite, Copper(I) iodide, DiOC6, Diphosphorus tetraiodide, Dithiazanine iodide, Echothiophate, Einsteinium(III) iodide, Eschenmoser's salt, Ethylenediamine dihydroiodide, Gallium(III) iodide, GelGreen, GelRed, Germanium iodide, Gold monoiodide, Gold triiodide, Hydrogen iodide, Iodine oxide, Iodomethylzinc iodide, Iodosilane, Iron(II) iodide, Lead(II) iodide, Lithium iodide, Magnesium iodide, Manganese(II) iodide, Mercury(I) iodide, Mercury(II) iodide, Nickel(II) iodide, Nitrogen triiodide, Palladium(II) iodide, Phosphorus triiodide, Polyiodide, Potassium iodide, Potassium tetraiodomercurate(II), Propidium iodide, Rubidium iodide, Rubidium silver iodide, Samarium(II) iodide, Silicon tetraiodide, Silver iodide, Sodium iodide, Strontium iodide, Tellurium iodide, Tellurium tetraiodide, Terbium(III) iodide, Tetraethylammonium iodide, Thallium triiodide, Thallium(I) iodide, Thorium(IV) iodide, Tibezonium iodide, Tiemonium iodide, Tin(II) iodide, Tin(IV) iodide, Titanium tetraiodide, Triiodide, Trimethylsilyl iodide, Trimethylsulfoxonium iodide, Uranium pentaiodide, Uranium tetraiodide, Uranium triiodide, Vanadium(III) iodide, Zinc iodide, and Zirconium(IV) iodide.

In particular embodiments, said halogen compound is an iodide comprising sodium iodide, potassium iodide, hydrogen iodide, calcium iodide, or silver iodide.

In some embodiments, said halogen compound is an iodate comprising one or more compounds from the non-limiting list of Calcium iodate, Iodic acid, Potassium iodate, Seeligerite, Silver iodate, and Sodium iodate.

In particular embodiments, said halogen compound is an iodate comprising sodium iodate, potassium iodate, calcium iodate, or silver iodate.

In some embodiments, said halogen compound is an organoiodide comprising one or more compounds from the non-limiting list of ²⁵I-NBF, ²⁵I-NBMD, ²⁵I-NBOH, ²⁵I-NBOMe, 2C-I, 5, 5-I-R91150, Acetrizoic acid, Adipiodone, Adosterol, Altropane, AM-1241, AM-2233, AM-630, AM-679 (cannabinoid), AM-694, AM251, Amiodarone, Benziodarone, Bromoiodomethane, Budiodarone, Butyl iodide, Carbon tetraiodide, Chiniofon, Chloroiodomethane, Clioquinol, Diatrizoic acid, Diiodohydroxypropane, Diiodohydroxyquinoline, Diiodomethane, 2,5-Dimethoxy-4-iodoamphetamine, Domiodol, Erythrosine, Ethyl iodide, Ethyl iodoacetate, Fialuridine, Fluoroiodomethane, Haloprogin, Herapathite, IAEDANS, Ibacitabine, IDNNA, Idoxifene, Idoxuridine, Iniparib, Iobenguane, Iobenzamic acid, Iobitridol, Iocarmic acid, Iocetamic acid, Iodamide, Iodixanol, Iodoacetamide, Iodoacetic acid, Para-Iodoamphetamine, Iodobenzamide, Iodobenzene, 2-Iodobenzoic acid, 19-Iodocholesterol, Iodocyanopindolol, Iodoform, 1-Iodomorphine, Iodophenol, Iodophenpropit, 4-Iodopropofol, Iodopropynyl butylcarbamate, Iodotrifluoroethylene, Iodoxamic acid, 2-Iodoxybenzoic acid, Iofetamine (123I), Ioflupane (123I), Ioglicic acid, Ioglycamic acid, Iomazenil, Iomeprol, Iopamidol, Iopanoic acid, Iopentol, Iopromide, Iopydol, Iotrolan, Iotroxic acid, Ioversol, Ioxaglic acid, Ioxilan, Ipodate sodium, Isopropyl iodide, Methiodal, Methyl iodide, Metrizamide, Metrizoic acid, Pentafluoroethyl iodide, Plakohypaphorine, N-Propyl iodide, Propyliodone, Rafoxanide, Rose bengal, RTI-121, RTI-229, RTI-353, RTI-55, SB-258,585, Sodium acetrizoate, Tiratricol, Trifluoroiodomethane, and Tyropanoic acid.

In particular embodiments, said halogen compound is an organoiodide. Organoiodine compounds are organic compounds that contain one or more carbon-iodine bonds. Almost all organoiodine compounds feature iodide connected to one carbon center. These are usually classified as derivatives of I⁻. Some organoiodine compounds feature iodine in higher oxidation states. Organoiodine compounds, often used as disinfectants or pesticides, include, e.g., iodoform (CHI₃), methylene iodide (CH₂I₂), and methyl iodide (CH₃I). In particular embodiment, the organoiodide is a polyiodoorganic compound. Polyiodoorganic compounds are sometimes employed as X-ray contrast agents, in fluoroscopy, a type of medical imaging. A variety of such polyiodoorganic compounds are available commercially; many are derivatives of 1,3,5-triiodobenzene and contain about 50% by weight iodine. In certain embodiments, the agent is soluble in water, non-toxic and/or readily excreted. A representative reagent is Ioversol, which has water-solubilizing diol substituents. Other organoiodine compounds include but are not limited to the two thyroid hormones thyroxine (“T₄”) and triiodothyronine (“T₃”). Marine natural products are rich sources of organoiodine compounds, including the recently discovered plakohypaphorines from the sponge Plakortis simplex.

The present invention also includes the use of compounds, e.g., drug compounds, into which an iodine is incorporated. For example, an iodine may be incorporated into existing drugs such as N-acetyl cysteine, standard pain relievers, and non-steroidal anti-inflammatory drugs, such as, e.g., aspirin, ibuprofen and naproxen. Most NSAIDs act as nonselective inhibitors of the enzyme cyclooxygenase (COX), inhibiting both the cyclooxygenase-1 (COX-1) and cyclooxygenase-2 (COX-2) isoenzymes.

In certain embodiments, said halogen compound is a polyiodide. The polyiodides are a class of polyhalogen anions composed of entirely iodine atoms. The most common and simplest member is the triiodide ion, I₃. Other known, larger polyiodides include [I₄]²⁻, [I₅]⁻, [I₇]⁻, [I₈]²⁻, [I₉]⁻, [I₁₀]²⁻, [I₁₀]⁴⁻, [I₁₁]⁻, [I₁₂]²⁻, [I₁₃]³⁻, [I₁₆]²⁻, [I₂₂]⁴⁻, [I₂₆]³⁻, [I₂₆]⁴⁻, [I₂₈]⁴⁻ and [I₂₉]³⁻. One example of a polyiodide is Lugol's iodine, also called Lugol's solution. Lugol's solution is commercially available in different potencies of 1%, 2%, or 5% Iodine. The 5% solution consists of 5% (wt/v) iodine (I₂) and 10% (wt/v) potassium iodide (KI) mixed in distilled water and has a total iodine content of 130 mg/mL. Potassium iodide renders the elementary iodine soluble in water through the formation of the triiodide (I⁻³) ion. Other names for Lugol's solution are 12KI (iodine-potassium iodide); Markodine, Strong solution (Systemic); and Aqueous Iodine Solution BCP. Examples of polyiodides, including their ions and counter-cations are shown in Table 1.

TABLE 1
Polyiodides

| Anion | Counter-cation |
|---|---|
| [I₃]⁻ | Cs⁺ |
| [I₄]²⁻ | [Cu(NH₃)₄]²⁺ |
| [I₅]⁻ | [EtMe₃N]⁺ |
| | [EtMePh₂N]⁺ |
| [I₇]⁻ | [Ag(18aneS₆)]⁺ |
| [I₈]²⁻ | [Ni(phen)₃]²⁺ |
| [I₉]⁻ | [Me₂ⁱPrPhN]⁺ |
| | [Me₄N]⁺ |
| [I₁₀]²⁻ | [Cd(12-crown-4)₂]²⁺ |
| [I₁₁]³⁻ | [(16aneS₄)PdIPd(16aneS₄)]³⁺ |
| [I₁₂]²⁻ | [Ag₂(15aneS₅)₂]²⁺ |
| | [Cu(Dafone)₃]²⁺ |
| [I₁₃]³⁻ | [Me₂Ph₂N]⁺ |
| [I₁₆]²⁻ | [Me₂Ph₂N]⁺ |
| | [ⁱPrMe₂PhN]⁺ |
| [I₂₂]⁴⁻ | [MePh₃P]⁺ |
| [I₂₆]³⁻ | [Me₃S]⁺ |
| [I₂₉]⁴⁻ | DMFc⁺ |
| [I₂₉]³⁻ | Cp₂Fe |
| [I₂₂]⁴⁻ | [MePh₃P]⁺ |
| [I₂₆]³⁻ | [Me₃S]⁺ |
| [I₂₆]⁴⁻ | DMFc⁺ |
| [I₂₉]³⁻ | Cp₂Fe |
| [I₂₂]⁴⁻ | [MePh₃P]⁺ |
| [I₂₆]³⁻ | [Me₃S]⁺ |
| [I₂₆]⁴⁻ | DMFc⁺ |

In one embodiment, the halogen compound is a tincture of iodine solutions, which comprises or consists of elemental iodine, and iodide salts dissolved in water and alcohol.

In some embodiments, said halogen compound is a periodate comprising one or more compounds from the non-limiting list of Dess-Martin periodinane, I, 2-Iodoxybenzoic acid, Periodic acid, Potassium periodate, and Sodium periodate.

In particular embodiments, said halogen compound is a periodate comprising sodium periodate, potassium periodate, calcium periodate, or silver periodate.

In particular embodiments, said halogen compound is a periodinane. Periodinanes are chemical compounds containing hypervalent iodine. In some embodiments, said halogen compound is a periodinane comprising one or more compounds from the non-limiting list of (Bis(trifluoroacetoxy)iodo)benzene, Dess-Martin periodinane, Iodobenzene dichloride, Iodosobenzene, and 2-Iodoxybenzoic acid.

In one embodiment, the halogen compound is an oil-infused iodide or iodine oil infusion.

In one embodiment, the present invention relates to a halogen compound containing bromine. Certain embodiments may comprise a bromine-containing halogen compound that is a bromide, bromate, organobromide, or a perbromate.

In some embodiments, said halogen compound is a bromide comprising one or more compounds from the non-limiting list of Aclidinium bromide, Aluminium bromide, Ammonium bromide, ANNINE-6plus, Antimony tribromide, Arsenic tribromide, Barium bromide, Benzododecinium bromide, Beryllium bromide, Bibenzonium bromide, Bismuth tribromide, Boron tribromide, Bromargyrite, Bromo(tetrahydrothiophene)gold(I), Bromopentaamminecobalt(III)bromide, Bromopentacarbonylrhenium(I), Cadmiumbromide, Caesium bromide, Caesium cadmium bromide, Calcium bromide, Cerium(III) bromide, Cetrimonium bromide, Chromium(III) bromide, Cimetropium bromide, Clidinium bromide, Cobalt(II) bromide, Copper(I) bromide, Copper(II) bromide, Cyanogen bromide, Demecarium bromide, Ditellurium bromide, DODAB, Domiphen bromide, EEthidium bromide, Fazadinium bromide, Fentonium, Gallium(III) bromide, Gold(I) bromide, Gold(III) bromide, Hexafluronium bromide, Hydrobromic acid, Hydrogen bromide, Indium(I) bromide, Indium(III) bromide, Iodine monobromide, Iron(II) bromide, Iron(III) bromide, Lanthanum(III) bromide, Lead(II) bromide, Lithium bromide, Magnesium bromide, Manganese(II) bromide, Mercury(I) bromide, Mercury(II) bromide, Morphine methylbromide, Nickel(II) bromide, Niobium bromide, Niobium(V) bromide, Nitrogen tribromide, Nitrosyl bromide, Otilonium bromide, Oxitropium bromide, Oxyphenonium bromide, Palladium(II) bromide, Pancuronium bromide, Phosphorus heptabromide, Phosphorus pentabromide, Phosphorus tribromide, Pifithrin, Pipecuronium bromide, Platinum(II) bromide, Platinum(IV) bromide, Polonium dibromide, Potassium bromide, Propantheline bromide, Radium bromide, Rubidium bromide, Silicon tetrabromide, Silver bromide, Sodium bromide, Strontium bromide, TTantalum(V) bromide, Tellurium tetrabromide, Terbium(III) bromide, Tetrabromoauric acid, Tetrabromomethane, Thallium(I) bromide, Timepidium bromide, Tin(II) bromide, Tin(IV) bromide, Titanium tetrabromide, Tribromosilane, Triphenylcyclopropenium bromide, Tungsten(V) bromide, Tungsten(VI) oxytetrabromide, Uranium pentabromide, Uranium tetrabromide, Vanadium(III) bromide, Ytterbium(III) bromide, Yttrium(III) bromide, Zinc bromide, and Zirconium(IV) bromide.

In particular embodiments, said halogen compound is a bromide comprising sodium bromide, potassium bromide, hydrogen bromide, calcium bromide, or silver bromide.

In some embodiments, said halogen compound is a bromate comprising one or more compounds from the non-limiting list of Bromic acid, Calcium bromate, Potassium bromate, Silver bromate, Sodium bromate, and Strontium bromate.

In some embodiments, said halogen compound is an organobromide comprising one or more compounds from the non-limiting list of 2-Bromobutyric acid, 25B-NBOMe, 2C-B, 2C-B-BZP, 2C-B-FLY, 2CB-Ind, 2CBCB-NBOMe, 2CBFly-NBOMe, 66-Br-APB, Acecarbromal, Ageliferin, Allyl bromide, AM-087, Ambroxol, Arbidol, AS-8112, BCDMH, Benzbromarone, Benzyl bromide, Bibrocathol, Brallobarbital, Bretazenil, Bretylium, Bretylium for the treatment of ventricular fibrillation, Brimonidine, Brivudine, Brodifacoum, Brodimoprim, Brofaromine, Bromacil, Bromadiolone, Bromadoline, Bromantane, Bromazepam, Bromazine, Bromethalin, Bromfenac, Bromhexine, Brominated flame retardant, Bromisoval, 2-Bromo-1-chloropropane, 4-Bromo-3,5-dimethoxyamphetamine, 2-Bromo-4,5-methylenedioxyamphetamine, Bromo-DragonFLY, Bromoacetic acid, Bromoacetone, Bromoacetylalprenololmenthane, 8-Bromoadenosine 3′,5′-cyclic monophosphate, Para-Bromoamphetamine, 4-Bromoaniline, Bromoanisole, Bromobenzene, Bromobimane, 1-Bromobutane, 2-Bromobutane, Bromochlorodifluoromethane, Bromochloromethane, Bromochlorosalicylanilide, Bromocresol green, Bromocresol purple, Bromocriptine, Bromocyclohexane, Bromodeoxyuridine, Bromodichloromethane, Bromodifluoroacetyl chloride, Bromodifluoromethane, Bromodiphenylmethane, B cont.Bromoethane, Bromofluoromethane, Bromoform, 3-Bromofuran, 8-Bromoguanosine 3′,5′-cyclic monophosphate, 1-Bromohexane, 2-Bromohexane, Bromoiodomethane, Bromomethane, 4-Bromo-N-methylcathinone, Bromopentane, Bromophenol blue, Bromadol, 2-Bromopropane, Bromopyruvic acid, N-Bromosuccinimide, Bromotrifluoromethane, 5-Bromouracil, 5-Bromouridine, Bromoxynil, Bromperidol, Brompheniramine, Bromsulphthalein, Bronidox, Bronopol, Brophebarbital, Bropirimine, Brotizolam, Broxaterol, Broxyquinoline, Butallylonal, Tert-Butyl bromide, C-8813, Carbromal, Chlorfenapyr, Ciclotizolam, Convolutindole A, DBDMH, DBNPA, Decabromodiphenyl ether, Deltamethrin, Desformylflustrabromine, Dexbrompheniramine, Diarylpyrimidines, 1,2-Dibromo-3-chloropropane, 1,4-Dibromobenzene, Dibromochloromethane, Dibromodifluoromethane, 1,1-Dibromoethane, 1,2-Dibromoethane, Dibromofluoromethane, Dibromomethane, 1,2-Dibromopropane, 1,3-Dibromopropane, Dibromotetrafluoroethane, Dibromotyrosine, Dibrompropamidine, Difethialone, 2,5-Dimethoxy-4-bromoamphetamine, DS-1 (drug), Ebrotidine, Embramine, Eosin, Eosin B, Eosin Y, Ethyl bromoacetate, Etravirine, FL3 (flavagline), Flubromazolam, Gidazepam, H-89, Halofuginone, Halomon, Halothane, Haloxazolam, Hexabromocyclododecane, Ibrolipim, Imidazenil, Isobromindione, JWH-249, JWH-424, KF-26777, Lonafarnib, Mebroqualone, Merbromin, Meta-DOB, Metaclazepam, Mitobronitol, Mucobromic acid, Narcobarbital, Nelotanserin, Neltenexine, NGD-4715, Nicergoline, O-806, Octabromodiphenyl ether, Organobromine compound, P7C3, Pamabrom, PEAQX, Pentabromodiphenyl ether, Phenacyl bromide, Phenazepam, 2-Phenylethylbromide, Phloxine, Pinaverium, Pindobind, Pipobroman, PNU-282,987, Polybrominated biphenyl, Polybrominated diphenyl ethers, Propallylonal, Propargyl bromide, N-Propyl bromide, Remoxipride, Romifidine, RTI-51, SB-357,134, Sigmodal, SSR-180,711, Stampidine, Surinabant, Surugatoxin, TCB-2, Tetrabromobisphenol A, Tetrabromoethane, Tetrabromoethylene, Tetrabromomethane, TH-302, Tilbroquinol, Tralomethrin, 2,4,6-Tribromoanisole, and Tribromofluoromethane.

In some embodiments, said halogen compound is a perbromate, said perbromate comprising sodium perbromate, potassium perbromate, hydrogen perbromate, or silver perbromate.

Particular embodiments of the present invention relate to a reduced form of a halogen compound. Many acceptable means of reduction of halogen compounds are possible and known to one skilled in the art. Examples of reduced forms of halogen compounds include halides, e.g., iodide and bromide, wherein the halogen has a valency of −1, including salt forms, such as NaI. Non-limiting examples of reduction methods include chemical reduction with electropositive elemental metals (such as lithium, sodium, magnesium, iron, zinc, and aluminum, e.g.), hydride transfer reagents (such as NaBH₄ and LiAIH₄, e/g), or the use of hydrogen gas with a palladium, platinum, or nickel catalyst.

A particular embodiment of the present invention relates to the administration of a halogen compound of the type described herein, e.g., a halide such as an iodide (e.g., NaI), to a mammalian subject, in a composition, concentration or formulation that is not significantly toxic to said mammals, e.g., a pharmaceutical composition. In particular embodiments, a halogen compound known to be toxic to a mammalian subject is excluded from the present invention. Thus, in particular embodiments, potassium iodide is excluded from the present invention. It is further contemplated that some embodiments may comprise the administration of more than one of said halogen compounds to said mammal, either simultaneously or separately, such that the combination of said compounds that are not individually significantly toxic are also not significantly toxic when combined.

Other compounds comprising a halogen compound or halogen element may also be used according to methods of and/or included in compositions of the present invention. In some embodiments, said halogen compound is a commercially available substance. In certain embodiments, said commercially available substances may include radiological contrast agents, topical iodine preparations, solutions, or drugs. In certain embodiments, said commercially available substance comprises iodine, and may be selected from the non-limiting list of Diatrizoate, Ipanoic acid, Ipodate, Iothalamate, Metrizamide, Diatrozide, Diiodohydroxyquinolone, Iodine tincture, Povidone iodine, Iodochlorohydroxyquinolone, Iodoform gauze, Saturated potassium iodide (SSKI), Lugol solution, Iodinated glycerol, Echothiopate iodide, Hydriodic acid syrup, Calcium iodide, Amiodarone, Expectorants, Vitamins containing iodine, Iodochlorohydroxyquinolone, Diiodohydroxyquinolone, Potassium iodide, Benziodarone, Isopropamide iodide, levothyroxine, and Erythrosine. In certain embodiments, said commercially available substance comprises bromine, and may be selected from the non-limiting list of Alphagen (brimonidine), Atrovent (Ipratropium), Celexa (citalopram), Combivent (ipratropium bromide), Enablex (darifenacin), Guaifenex DM (dextromethorphan), Razadyne (galantamine), and Spiriva (tiotropium).

Compositions and Unit Dosage Forms

The present invention also includes compositions comprising a halogen compound (e.g., an iodide or bromide). In particular embodiments, the compositions are pharmaceutical compositions comprising a halogen compound and one or more pharmaceutically acceptable carriers, diluents or excipients, e.g., a buffer. In particular embodiments, the composition further comprises one or more additional active agents. In certain embodiments, compositions of the present invention are pharmaceutical compositions comprising a halogen compound, optionally a halide, e.g., an iodide, such as NaI. In certain embodiments, the compositions comprise a reduced form of a halogen compound, i.e., a halogen in a −1 valence state. In particular embodiments, the reduced form of a halogen is a reduced form of iodine, such as iodide. In particular embodiments, the compound containing a reduced form of iodine is NaI, KI, HI, Cal or AgI.

In certain embodiments, the present invention includes compositions comprising a halogen compound, e.g., a reduced form of a halogen compound. In certain embodiments of any of the compositions of the invention, the halogen compound is a reduced form of a halogen compound, which comprises a halogen in a −1 valence state, e.g., an iodide or bromide, such as sodium iodide. The reduced form of halogen compound may be any of those described herein. In certain embodiments of any of the compositions described herein, the composition further comprises glutathione or another reducing agent. In particular embodiments, a composition comprises glutathione and iodide. In particular embodiments, at least a portion of the iodide or iodate is in reduced form, and the glutathione inhibits the oxidation of the halogen compound in the composition.

In particular embodiments, the compositions are formulated to maintain the halogen in a reduced form when stored over a period of time. Thus, the compositions may be stable compositions of reduced forms of halogen compounds or salts or precursors thereof, whose effectiveness as a therapeutic may normally be compromised during manufacture and storage, as a result of oxidation reactions that produce oxidation products. The compositions of the present invention have increased shelf-life, are easily and reproducibly manufactured, are designed for standard routes of administration, and are, therefore, advantageous in the treatment and prevention of a number of diseases, conditions and injuries. In certain embodiments, a stable composition comprising a halogen compound comprises glutathione or another reducing agent.

In certain embodiments of the compositions, a composition is considered stable, i.e., a stable composition, if at least 90% of the halogen compound in the composition is present in reduced form for at least one hour either when stored at room temperature, 4° C., 25° C., 40° C. or 50° C. In related embodiments, a composition is considered stable if at least 70%, at least 80%, at least 91%, at least 92%, at least 93%, at least 94%, at least 95%, at least 96%, at least 97%, at least 98%, or at least 99% of the halogen compound in the composition is present in reduced form for at least one hour either when stored at room temperature or when stored at 4° C. In certain embodiments of the stable compositions, at least 90% of the halogen compound in said composition is present in said reduced form for at least one day, at least one week, at least one month, at least two months, at least four months, at least six months, or at least one year, either when stored at room temperature or when stored at 4° C., 25° C., 40° C. or 50° C. In related embodiments, at least 70%, at least 80%, at least 91%, at least 92%, at least 93%, at least 94%, at least 95%, at least 96%, at least 97%, at least 98%, or at least 99% of the halogen compound in the stable composition is present in said reduced form for at least one day, at least one week, at least one month, at least two months, at least four months, at least six months, or at least one year, either when stored at room temperature or when stored at 4° C. In particular embodiments, at least 98% of the halogen compound in the stable composition is present in said reduced form for at least one month or at least six months when stored at 4° C. In related embodiments, at least 70%, at least 80%, at least 91%, at least 92%, at least 93%, at least 94%, at least 95%, at least 96%, at least 97%, at least 98%, or at least 99% of the halogen compound in the stable composition is present in said reduced form for at least one day, at least one week, at least one month, at least two months, at least four months, at least six months, or at least one year, either when stored at room temperature or when stored at room temperature or 25° C. In particular embodiments, at least 98% of the halogen compound in the stable composition is present in said reduced form for at least one month or at least six months when stored at room temperature or 25° C. In related embodiments, at least 70%, at least 80%, at least 91%, at least 92%, at least 93%, at least 94%, at least 95%, at least 96%, at least 97%, at least 98%, or at least 99% of the halogen compound in the stable composition is present in said reduced form for at least one day, at least one week, at least one month, at least two months, at least four months, at least six months, or at least one year, either when stored at room temperature or when stored at 40° C. or 50° C. In particular embodiments, at least 98% of the halogen compound in the stable composition is present in said reduced form for at least one month or at least six months when stored at 40° C. or 50° C. In various embodiments, the composition is a liquid pharmaceutical composition, while in other embodiments, the composition is a solid or powder, or is dried, lyophilized, or freeze-dried.

In particular embodiments, the present invention relates to a stable liquid composition comprising iodide, wherein the stable liquid composition comprises less than 1% of any of the following oxidation products of iodide (−1 oxidation state): hypoiodite (+1 oxidation state), iodite (+3 oxidation state), iodate (+5 oxidation state), or periodate (+7 oxidation state). In particular embodiments, the stable liquid composition comprising iodide comprises less than 1% iodine (I₂).

In particular embodiments, any of the compositions described herein comprise a pharmaceutically acceptable carrier, diluent or excipient. Further, any of the compositions may comprise one or more of a buffer, a reducing agent, a tonicity agent, a stabilizer, a surfactant, a lycoprotectant, a polyol, an antioxidant, or a preservative. In particular embodiments, any of the compositions described herein comprise glutathione.

In particular embodiments, compositions may comprise one or more solvents. In particular embodiments, the solvent is water. In particular embodiments, the solvent is a phosphate-buffered saline.

Compositions of the present invention and methods of the present invention may include a halogen compound, or salt or precursor thereof, in any desired concentration. The concentration may be readily optimized, e.g., depending upon the type of injury or disease being treated and the route of administration, so as to deliver an effective amount in a convenient manner and over an appropriate time-frame.

In some embodiments, the concentration of halogen compound or salt or precursor thereof (e.g., iodide, such as NaI) present in a composition of the present invention is about 0.0001 mM to about 100 M, about 0.0005 mM to about 50 M, about 0.001 mM to about 10 M, about 0.001 mM to about 5 M, about 0.001 mM to about 1 M, about 0.005 mM to about 10 M, about 0.005 mM to about 5 M, about 0.005 mM to about 1 M, about 0.005 mM to about 0.5 M, about 0.01 mM to about 10 M, about 0.01 mM to about 5 M, about 0.01 mM to about 2 M, about 0.1 mM to about 1 M, about 0.1 mM to about 0.5 M, about 0.5 mM to about 5 M, about 0.5 mM to about 2 M, about 0.5 mM to about 1 M, about 0.5 mM to about 0.5 M, about 1 mM to about 5 M, about 1 mM to about 2 M, about 1 mM to about 1 M, about 1 mM to about 0.5 M, about 5 mM to about 5 M, about 5 mM to about 2 M, about 5 mM to about 1 M, about 5 mM to about 0.5 M, about 5 mM to about 0.25 M, about 10 mM to about 1 M, about 10 mM to about 0.5 M, about 10 mM to about 0.25 M, or about 10 mM, about 50 mM about 100 mM, or about 200 mM.

As used herein, the term “%” when used without qualification (as with w/v, v/v, or w/w) means % weight-in-volume for solutions of solids in s (w/v), % weight-in-volume for solutions of gases in s (w/v), % volume-in-volume for solutions of s in s (v/v) and weight-in-weight for mixtures of solids and semisolids (w/w) (Remington's Pharmaceutical Sciences (2005); 21st Edition, Troy, David B. Ed. Lippincott, Williams and Wilkins).

In certain embodiments, a composition comprises glutathione at a concentration of about 1.5 μM to about 10 M, about 15 μM to about 1 M, about 150 μM to about 1 M, about 1.5 mM to about 1 M, about 10 mM to about 500 mM, about 10 mM to about 250 mM, or about 100 mM, about 120 mM, about 150 mM, about 170 mM, or about 200 mM.

In certain embodiments, a composition of the invention comprises a halogen compound (e.g., iodide, such as NaI), and optionally glutathione, wherein the concentration of glutathione is about 100 μM to about 1 M, about 1 mM to about 1 M, or about 10 mM to about 500 mM, and, the concentration of halogen compound is about 0.01 mM to about 5 M, about 1 mM to about 0.5 M, or about 10 mM to about 250 mM. In particular embodiments, the halogen compound is an iodide. In particular embodiments of any of these compositions, the composition is formulated for oral delivery, or is an oral dosage form, the halogen compound (when present) comprises iodine (e.g., iodide). In particular embodiments, the composition is formulated for intravenous administration, and the halogen compound (if present) is iodide. In one embodiment, the composition comprises iodide and glutathione, each within any of the concentration ranges or at a concentration described herein.

In particular embodiments, the pH of a composition of the present invention is in the range of (3.0-12.0), while in other embodiments, the pH is in the range of (5.0-9.0). The pH of the pharmaceutical composition may be adjusted to a physiologically compatible range. For example, in one embodiment, the pH of the stable composition is in the range of 6.5-8.5. In other embodiments, the compositions of the present invention have a pH in the range of 7.5-8.5 or 7.4-9.0.

In particular embodiments, oxygen is present in a composition of the present invention at a concentration in the range of 0 M⁻⁵ μM or 0 μM⁻¹ μM or 0 μM⁻¹ μM or 0 μM-0.01 μM. In particular embodiments, oxygen is present in the composition at a concentration of less than 3 M, less than 1 M, less than 0.1 μM, less than 0.01 μM, or less than 0.001 μM.

In certain embodiments, the compositions of the present invention may further comprise a limited amount of oxidation products. Oxidation products that may be present in various embodiments of the present invention include, but are not limited to, iodine, iodate, bromine, and bromate. In various embodiments, one or more of these oxidation products is present in a composition in an amount less than 10%, less than 5.0%, less than 2.0%, less than 1.0%, less than 0.5%, less than 0.2%, less than 0.1%, less than 0.05%, or less than 0.01% (w/v) of the total halogen compound in the composition.

In one embodiment, a composition has an osmolarity in the range of 200-400 mOsmol/L. NaCl may be used as an excipient to adjust osmolality.

In certain embodiments, isotonicity of the compositions is desirable as it results in reduced pain upon administration and minimizes potential hemolytic effects associated with hypertonic or hypotonic compositions. Thus, the compositions of the invention not only have increased storage stability, but also have the added benefit of substantially reduced pain upon administration when compared with formulations using other more traditional buffer systems consisting of an acid and a salt form of the acid.

In particular embodiments, the liquid is sodium hydroxide.

In certain embodiments, the composition has a pH in the range of 6.5 to 8.5 and has an oxygen content of less than or equal to 5 M for 3 months when stored within a temperature range of 23°-27° or 6 months when stored at a temperature range of (23°-27°). In one embodiment, the composition has an osmolarity in the range of 250-330 mOsmol/L. It may be isotonic or near isotonic.

The present invention further includes kits comprising composition(s) of the present invention. In certain embodiments, such kits comprise one or more containers to store the composition(s) of the present invention. In one embodiment, a composition is stored in the container under an inert or noble gas, and the container is a sealed and has an oxygen impermeable light-protective container (e.g., an amber vial). In certain embodiments, a kit comprises a first pharmaceutical composition comprising a halogen compound, e.g., a reduced form of iodine, such as iodide.

In certain embodiments, a composition is packaged in an impermeable container. “Impermeable container” refers to containers that provide a barrier to the passage of gas molecules. Impermeable containers are known to those skilled in the art and include, but are not limited to, “i.v. bags” or syringes comprising a gas impermeable construction material, or a sealed glass vial. In particular embodiments, the composition may be packaged into an impermeable container containing an inert atmosphere, an inert gas, or a noble gas. Noble gas refers to helium (He), neon (Ne), argon (Ar), krypton (Kr), xenon (Xe), and radon (Rn). Inert gas refers to nitrogen (N2). The term “inert atmosphere” refers to a nitrogen or argon atmosphere in a container. In particular embodiments, the container comprises a reduced oxygen or oxygen-free environment. A “reduced oxygen environment” is an environment having an oxygen concentration of less than 100 parts per million. The composition may be packaged in a light-protective vial or container, e.g., amber vials. In one embodiment, the composition is sealed and stored in a glass ampoule.

In some embodiments, compositions of the present invention comprise one or more excipients included to prevent oxidation of the halogen compound during storage, where storage is in the range of one to twelve months or longer. In some embodiments, storage is in the range of one to six months. In some embodiments, storage is in the range of three to six months. In some embodiments, storage is in the range of four to five months. Embodiments of the present invention may use a single excipient or a combination of excipients. There are many suitable excipients. Examples include chelators, pH modifying agents, reducing agents, antioxidants, spin-trap agents and preservatives.

Compositions of the present invention may further comprise one or more pH modifying agents. pH modifying agents, include, but are not limited to, inorganic salts, such as zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, sodium chloride, sodium carbonate, sodium bicarbonate, potassium hydroxide, potassium phosphate, sodium bicarbonate, thioglycolic acid, zinc acetate, zinc hydrogen phosphate, zinc phosphate, zinc lactate, zinc maleate, zinc oleate, zinc oxalate, and combinations thereof. Other pH modifying agents include, e.g., acetic acid, fumaric acid, malic acid, nitric acid, phosphoric acid, propionic acid, sulfuric acid, tartaric acid, carbon dioxide, carbonic acid, N-methyl-D-glucamine, 4-(2-hydroxyethyl)-morpholine, Tromethamine, Orotic acid, and hydrochloric acid. In one embodiment, the pH modifying agent is sodium hydroxide.

A pH modifying agent may serve as a buffering agent when it is added to an already acidic or basic solution, which it then modifies and maintains at a new pH (see: The United States Pharmacopeia-National Formulary 29th Edition, (2006) Rockville, Md.; Stahl, P. Wermuth, C. ed. Handbook of Pharmaceutical Salts Properties, Selection and Use. Wiley (2002)).

In certain embodiments, compositions of the present invention include one more excipients that are reducing agents, such as, e.g., glutathione (see: U.S. Pat. No. 6,586,404), tris(2-carboxyethyl) phosphine hydrochloride (TSEP), thiosulfate, I-cysteine, cysteine or methionine. In one embodiment, the reducing agent is glutathione (see: Vincent et al., Endocrine Reviews (2004) 25:612-628), dithiothreitol (DTT) (Weir et al., Respir and Physiol Biol; (2002) 132:121-30) or dithioerythritol (DTE). In certain embodiments, the concentration of glutathione is about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or M or more or any range derivable therein. In certain embodiments, the concentration of dithiothreitol (DTT), which present at about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or 1 M, or any range derivable therein. In certain embodiments, the reducing agent is dithioerythritol (DTE), is about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or M, or any range derivable therein.

Compositions of the present invention may optionally comprise a free radical scavenger or antioxidant. Examples of free radical scavengers or antioxidants include, but are not limited to, ascorbic acid (vitamin C), D-alpha tocopherol acetate, DL-alpha-tocopherol (vitamin E), melatonin, sodium bisulfite, sodium sulfite, sodium metabisulfite, Trolox (6-hydroxy-2,5,7,8-tetramethyl chroman-2-carboxylic acid), Tris(2-Carboxyethyl) phosphine Hydrochloride (TCEP), melatonin, dithionite, pyrosulfite, cysteine, potassium disulfite, sodium thioglycolate, thioethylene glycol, L-threoascobic acid, acetylsalicylic acid, salicylic acid, lecithin, ascorbyl palmitate, butylated hydroxyanidole, ascorbic acid, butylated hydroxyanisole, butylated hydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, lecithin, ethanolamine, meglumine and combinations thereof (see US 2005/0106214). In one embodiment, the anti-oxidant agent is a spin-trap agent. Examples of spin-trap agents include, but are not limited to, N-t-butyl-phenylnitrone (PBN) (see: Kotake, Y., Antioxid Redox Signal (1999) 481), 4-Hydroxy-2,2,6,6-tetramethylpiperidine-1-oxyl (TEMPOL) (Gariboldi, M. B., et al. (2000), Free Radic. Biol. Med. 29:633; Miura, Y., et al. J. Radiat. Res. (Tokyo) (2000) 41:103; Mota-Filipe, H., et al. (1999), Shock 12:255R: 22-41; S: 39-26 2,2,6,6-tetramethylpiperidine-N-oxyl (TEMPO) (see: Lapchak, et al., Stroke (2001) 32:147-53); (disodium-[(tert-butylimino) methyl]benzene-1,3-disulfonate N-oxide (NXY-059) (see: Lapchak et al., CNS Drug Rev (2003) 9:253-62). In some embodiments, the spin-trap agent is TEMPO, which is present in the range of 0 mg/kg-1,000 mg/kg. In some embodiments, the spin-trap agent is TEMPO and is present in the range of 100 mg/kg-1,000 mg/kg. In another embodiment, the spin-trap agent is TEMPO and is present in the range of 0 mg/kg-100 mg/kg.

Compositions of the present invention may optionally comprise a preservative. As used herein, the term “preservative” is intended to mean a compound used to prevent the growth of microorganisms.

The present invention also includes unit dosage forms of compositions of the present invention. In certain embodiments, the unit dosage form comprises or consists of an effective amount of a halogen compound, e.g., iodide, for treating, reducing the severity or duration of, or preventing PICS. In certain embodiments, a unit dosage form further comprises glutathione in an amount effective to maintain the halogen compound in a reduced form under any of the conditions described herein. In particular embodiments, the unit dosage form is formulated for intravenous administration, administration by infusion, or oral administration.

In particular embodiments, a unit dosage form comprising a halogen compound, such as an iodide or NaI, comprises or consists of about 0.005 mg to about 5000 mg, about 0.05 to about 1000 mg, about 0.5 mg to about 100 mg, about 1 mg to about 100 mg, about 2.5 mg to about 100 mg, about 0.5 mg to about 50 mg, about 1 mg to about 50 mg, about 2.5 mg to about 50 mg, about 5 mg to about 50 mg, about 10 mg to about 50 mg, or about 1 mg, about 2 mg, about 5 mg, about 10 mg, or about 15 mg. In certain embodiments, the unit dosage form comprises between about 1 mg and about 150 mg (including any interval in this range), between about 1 mg and about 125 mg, between about 1 mg and about 100 mg, between about 1 mg and about 75 mg, between about 1 mg and about 50 mg, between about 1 mg and about 25 mg or between about 1 mg and about 10 mg of the halogen compound. In certain embodiments, the unit dosage form comprises about 150 mg, about 125 mg, about 100 mg, about 75 mg, about 50 mg, about 25 mg or about 10 mg of the halogen compound. In certain embodiments, the unit dosage form comprises or a subject between about 50 mg and 500 mg, about 50 mg and 100 mg, about 100 mg and about 1000 mg (including any interval in this range), between about 150 mg and about 800 mg, between about 200 mg and about 700 mg, between about 250 mg and about 600 mg, between about 300 mg and about 500 mg, between about 350 mg and about 450 mg or between about 300 mg and about 700 mg of the halogen compound. In certain embodiments, the unit dosage form comprises about 200 mg, about 300 mg, about 400 mg, about 500 mg, about 600 mg, about 700 mg, about 800 mg, about 900 mg, or about 1000 mg of the halogen compound. In certain embodiments, the unit dosage form comprises less than or equal to 1000 mg, less than or equal to 800 mg, less than or equal to 700 mg, less than or equal to 500 mg, less than or equal to 250 mg, less than or equal to 200 mg, or less than or equal to 150 mg of the halogen compound. In related embodiments, the unit dosage form comprises less than or equal to 150 mg, less than or equal to 125 mg, less than or equal to 100 mg, less than or equal to 75 mg, less than or equal to 50 mg, less than or equal to 25 mg, or less than or equal to 10 mg of the halogen compound. In related embodiments of methods disclosed herein, the subject in need thereof is administered an amount of halogen compounds, e.g., iodide, such as NaI, that falls within any of these ranges or values.

In some embodiments, including, e.g., embodiments where the unit dosage form is formulated as a liquid, e.g., for intravenous administration or administration by infusion, the concentration of halogen compound or salt or precursor thereof present in a unit dosage form of the present invention is about 0.0001 mM to about 100 M, about 0.0005 mM to about 50 M, about 0.001 mM to about 10 M, about 0.001 mM to about 5 M, about 0.001 mM to about 1 M, about 0.005 mM to about 10 M, about 0.005 mM to about 5 M, about 0.005 mM to about 1 M about 0.005 mM to about 0.5 M, about 0.01 mM to about 10 M, about 0.01 mM to about 5 M, about 0.01 mM to about 2 M, about 0.1 mM to about 1 M, about 0.1 mM to about 0.5 M, about 0.5 mM to about 5 M, about 0.5 mM to about 2 M, about 0.5 mM to about 1 M, about 0.5 mM to about 0.5 M, about 1 mM to about 5 M, about 1 mM to about 2 M, about 1 mM to about 1 M, about 1 mM to about 0.5 M, about 5 mM to about 5 M, about 5 mM to about 2 M about 5 mM to about 1 M, about 5 mM to about 0.5 M, about 5 mM to about 0.25 M, about 10 mM to about 1 M, about 10 mM to about 0.5 M, about 10 mM to about 0.25 M, or about 10 mM, about 50 mM about 100 mM, or about 200 mM. The unit dosage form may further comprise one or more pharmaceutically acceptable diluents, excipients or carriers.

In certain embodiment, the unit dosage form comprises iodide, e.g., NaI, and the effective amount is greater than or equal to about 150 μg, greater than or equal to about 300 μg, greater than or equal to about 500 μg, greater than or equal to about 1 mg, greater than or equal to about 2 mg, greater than or equal to about 5 mg, greater than or equal to about 10 mg, greater than or equal to about 15 mg, or greater than or equal to about 20 mg. In certain embodiments, the effective amount is 150 μg to 1000 mg, 300 μg to 1000 mg, 500 μg to 1000 mg, 1 mg to 1000 mg, 2 mg to 1000 mg, 5 mg to 1000 mg, 10 mg to 1000 mg, 150 μg to 100 mg, 300 μg to 100 mg, 500 μg to 100 mg, 1 mg to 100 mg, 2 mg to 100 mg, 5 mg to 100 mg, or 10 mg to 100 mg.

In certain embodiments, a subject is administered an effective amount of halide, e.g., NaI, where the effective amount is between about 0.1 mg/kg to about 100 mg/kg, about 0.1 mg/kg to about 10 mg/kg, about 0.5 mg/kg to about 5 mg/kg, about 0.2 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg or about 10 mg/kg. In certain embodiments, the effective amount is 150 μg to 50 mg, 300 μg to 20 mg, 500 μg to 10 mg, 1 mg to 20 mg, 1 mg to 10 mg, or about 5 mg, about 10 mg, about 15 mg, or about 20 mg.

In other embodiments, the effective amount is between about 1 mg and about 150 mg (including any interval in this range), between about 1 mg and about 125 mg, between about 1 mg and about 100 mg, between about 1 mg and about 75 mg, between about 1 mg and about 50 mg, between about 1 mg and about 25 mg or between about 1 mg and about 10 mg of the halogen compound. In certain embodiments, the effective amount is about 150 mg, about 125 mg, about 100 mg, about 75 mg, about 50 mg, about 25 mg or about 10 mg of the halogen compound. In certain embodiments, the effective amount comprises less than or equal to 1000 mg, less than or equal to 800 mg, less than or equal to 700 mg, less than or equal to 500 mg, less than or equal to 250 mg, less than or equal to 200 mg, or less than or equal to 150 mg of the halogen compound. In certain embodiments, the effective amount is between about 100 mg and about 1000 mg (including any interval in this range), between about 150 mg and about 800 mg, between about 200 mg and about 700 mg, between about 250 mg and about 600 mg, between about 300 mg and about 500 mg, between about 350 mg and about 450 mg or between about 300 mg and about 700 mg of the halogen compound. In certain embodiments, the effective amount is about 200 mg, about 300 mg, about 400 mg, about 500 mg, about 600 mg, about 700 mg, about 800 mg, about 900 mg, or about 1000 mg of the halogen compound. In particular embodiments, the effective amount is the amount per day.

In certain embodiments, a composition of the invention may be formulated in a dosage form suitable for oral or parenteral administration. In addition, in particular embodiments, a composition of the invention may be in the form of an immediate or modified release formulation. For example, formulations of the halogen compound can be used to provide controlled release, in which the release of the compound(s) is controlled and regulated to allow less frequency of dosing or to improve the pharmacokinetic or toxicity profile of a given active agent.

In general the amount of the active compound present in a composition or unit dosage form depends inter alia on the specific compound and formulation, the age and condition of the subject, and the specific features of the injury, condition, or disease being treated or prevented, the route of administration and the dosage frequency.

The dosage frequency also depends on the injury, condition or disease being treated or prevented, the amount or concentration of the compound, the specific composition used, the route of administration and may incorporate subject-specific variation including, but not limited to age, weight, gender, or overall health.

In certain embodiments, a unit dosage form suitable for oral administration is in the form of a pill, drenches (aqueous or non-aqueous solutions or suspensions), boluses, powders, granules, polymer release formulations, pastes for application to the tongue tablet, caplet or a capsule. A pill is a small, round, solid pharmaceutical oral dosage form that was in use before the advent of tablets and capsules. In colloquial usage, tablets, capsules, and caplets are still often referred to as “pills” collectively. In certain embodiments, pills are made by mixing the active ingredients with an excipient such as glucose syrup in a mortar and pestle to form a paste, then divided into suitable sizes, and often coated with sugar to make them more palatable.

Dosage levels of a halogen compound present in a composition described herein may be varied as so to obtain an amount of the halogen compound that is effective to achieve the desired therapeutic effect for a particular subject, halogen compound and mode of administration, without being toxic to the subject.

In various embodiments, compositions and unit dosage forms of the invention may be formulated in any different manner suitable for a desired delivery route. Typically, formulations include all physiologically acceptable compositions. Such formulations may include a halogen compound, optionally in combination with one or more additional active agents, in combination with any physiologically acceptable carrier, diluent or excipient. Halogen compounds may be formulated for administration with any biologically acceptable medium, including but not limited to water, buffered saline, polyol, or mixtures thereof. “Biologically acceptable medium” includes any and all solvents, dispersion media, and the like that may be appropriate for the desired route of administration of the pharmaceutical composition. Suitable biologically acceptable media and their formulations are described, for example, in the most recent Remington's Pharmaceutical Sciences (Remington's Pharmaceutical Sciences. Mack Publishing Company, Easton, Pa., USA 1985).

Formulations, and unit dosages forms thereof, may contain suitable physiologically acceptable carriers comprising excipients and/or auxiliaries that facilitate processing of the halogen compound and/or other active agent into preparations that can be used pharmaceutically. Formulations, and unit dosage forms thereof, may also include agents that increase or otherwise affect the bioavailability of the halogen compound and/or other active agent. As used herein, “bioavailability” refers to the effect, availability and persistence of the active agent(s) after being administered to a subject.

Pharmaceutically acceptable carriers can be any pharmaceutically acceptable material, composition, or vehicle, including but not limited to a liquid or solid filler, diluent, excipient, solvent or encapsulating material, involved in carrying or transporting the subject agonists to an organ, or portion of the body.

The present invention further includes stable liquid pharmaceutical compositions formulated for parenteral administration, e.g., intravenous administration or administration by infusion. In certain embodiments, the stable liquid pharmaceutical compositions comprise a reduced form of a reduced form of a halogen compound. In particular embodiments, the composition further comprises glutathione. In particular embodiments, a stable liquid pharmaceutical compositions formulated for parenteral administration comprises a halogen compound, e.g., iodide, and glutathione. In particular embodiments, a stable liquid pharmaceutical compositions formulated for parenteral administration comprises iodide, and glutathione. The iodide and glutathione may be present at a concentration described herein, or in amount sufficient or appropriate to deliver an amount described herein to a subject. The concentration of each active agent in the composition may be readily determined based on the desired amount of each active agent to be delivered to a subject in need thereof. In particular embodiments, the composition comprises iodide, e.g., NaI, at a concentration of about 1 mM to about 1M or about 10 mM to about 500 mM, and glutathione at a concentration of about 1 mM to about 500 mM or about 10 mM to about 500 mM. In particular embodiments, the composition is contained within an oxygen-impermeable container, and may be under nitrogen or argon gas. In particular embodiments, the amount of composition present in the container is a unit dosage amount comprising or consisting of a suitable dosage amount for administration to a subject in need thereof.

Formulations of halogen compound for parenteral administration may comprise a halogen compound in combination with one or more pharmaceutically acceptable isotonic aqueous or nonaqueous solutions, dispersions, suspensions or emulsions, or powders which may be reconstituted into sterile injectable solutions or dispersions just prior to use. Parenteral formulations may contain antioxidants; buffers or solutes which render the formulation isotonic with the blood of the intended subject; bacteriostats; suspending; or thickening agents.

The present invention further includes kits comprising compositions or unit dosage forms of the present invention. In certain embodiments, such kits comprise one or more containers to store the compositions of the present invention. In one embodiment, the composition is stored in the container under an inert or noble gas, and the container is a sealed and has an oxygen impermeable light-protective container (e.g., an amber vial).

Compositions comprising halogen compounds, including reduced forms of halogen compounds, such as iodide and bromide, may be prepared by any means known and available. In certain embodiments, a halogen compound is dissolved in water or a suitable buffer, such as a NaCl buffer.

In certain embodiments, once produced, in various embodiments, a composition is stored in an impermeable container, e.g., an oxygen impermeable container. This is particularly desirable to prevent oxidation of the reduced form of halogen compound. Impermeable containers are known to those skilled in the art and include, but are not limited to, “i.v. bags” comprising a gas impermeable construction material, or a sealed glass vial. In particular embodiments, the impermeable container comprises an oxygen impermeable material having an oxygen transmission coefficient less than 10⁻¹⁰ [cm³(STP)/cm/(cm²+s+Pa)], wherein STP=standard temperature and pressure (25 degrees centigrade and pressure 1 atmosphere); PA=pascals, and s=second. For example, the walls of the container may comprise a layer of an oxygen impermeable polymer. Exemplary oxygen impermeable polymers include but are not limited to: silicon rubber, natural rubber, low density poly ethylene (LDPE), polystyrene (PS), polyethylene (PE), polycarbonate (PC), polyvinyl acetate (PVAc), amorphous polyethylene terephthalate (APET), polyvinly chloride (PVC), nylon 6 (Ny6), polyvinyl fluoride (PVF), polyvinylidene chloride (PVdC), polyacetonitrile (PAN), ethylene vinyl alcohol (EVOH), and polyvinyl alcohol (PVA). In certain embodiments, the oxygen transmission coefficient of said polymer is less than 10⁻¹⁰ [cm³(STP)/cm/(cm²+s+Pa)]. In particular embodiments, the walls of the container comprise multiple layers of one or more oxygen impermeable polymers.

To prevent exposure to air in the gas-tight storage container, an inert or noble gas, such as nitrogen or argon, may be introduced into a container containing a composition of the present invention prior to closure.

In other related embodiments, compositions are stored in a light-resistant or a light-protective container or vial, such as an amber vial. The composition may be packaged in a glass vial. It may be filled to a slight over-pressure in an inert atmosphere, e.g., nitrogen, to prevent/slow oxidative breakdown of the composition, and may be contained in a form such that ingress of light is prevented, thereby preventing photochemical degradation of the composition. This may be achieved using an amber vial. Additional container systems that permit a solution to be stored in an oxygen-free environment are known, as many intravenous solutions are sensitive to oxygen. For example, a glass container that is purged of oxygen during the filling and sealing process may be used. In another embodiment, flexible plastic containers are available that may be enclosed in an overwrap to seal against oxygen. Basically, any container that prevents oxygen from interacting with the stable composition may be used (see, e.g., U.S. Pat. No. 6,458,758). In one embodiment, the container includes one or more oxygen scavenger. For example, the oxygen scavenging composition can be applied as a coating or lining upon the inside surface of the product supporting or retaining means to function as a barrier to oxygen permeation (see, e.g., U.S. Pat. No. 5,492,742).

In particular embodiments, a container or vial may comprise a unit dosage of a composition of the present invention. In certain embodiments, the unit dosage form comprises or consists of an effective amount of the composition to treat or prevent a disease, condition, or injury, including any of those described herein, in a subject.

In particular embodiments, the present invention includes a container, such as a saline bag, that includes a premixed liquid composition of a halogen e.g., an iodide or bromide, wherein the amount of premixed liquid composition constitutes a dosage useful in treating or preventing a disease, condition, or injury, including any of those described herein, in a subject in need thereof, and one or more pharmaceutically acceptable carriers, diluents, or excipient. In particular embodiments, the liquid composition is sterile.

In particular embodiments, the present invention includes a container, such as a vial, that includes a dry composition of a halogen compound, e.g., an iodide or bromide, wherein the amount of dry composition constitutes a dosage useful in treating or preventing a disease, condition, or injury, including any of those described herein, in a subject in need thereof. The dry composition may be reconstituted, e.g., with a pharmaceutically acceptable carrier, diluent, or excipient, e.g., sterile water, prior to delivery to a subject in need thereof.

Methods of Using Halogen Compounds

The present invention includes, inter alia, methods and compositions related to the use of a halogen compound, e.g., I-, to treat, inhibit, reduce the severity of, or prevent secondary injury or damage to a subject resulting from a different primary injury, disease, disorder, or medical treatment. In particular embodiments, the secondary injury or damage occurs at least at an anatomical location distal to or remote from the location of the primary injury, disease, disorder, or medical treatment, e.g., a different organ or tissue. In the case of certain injuries or diseases, the secondary injury or damage may occur within a certain anatomical location within the subject, which, in certain embodiments, may be limited to a specific tissue or organ. In particular embodiments, the secondary injury or damage occurs at a time after the occurrence of the primary injury, disease, disorder, or medical treatment. In certain embodiments, a time period passes after the primary injury, disease, disorder, or medical treatment occurs and before the secondary injury or damage occurs. For example, the time period may be about 1 hour, about 4 fours, about 8 hours, about 12 hours, about 1 day, about 2 days, about 4 days, about 1 week, about 2 weeks, about 3 weeks, or about 1 month. In certain embodiments, the secondary injury or tissue damage results from a systemic inflammatory response initiated or increased or exasperated by the primary injury or disease.

Tissue Injury

In some embodiments, the present disclosure provides a method for treating, reducing the severity of, or preventing remote and/or local tissue injury or damage resulting from a primary injury or disease in a subject in need thereof, comprising providing to said subject a pharmaceutical composition comprising a halogen compound and a pharmaceutically acceptable carrier, diluent, or excipient. In certain embodiments, the primary injury or disease is localized to one or more regions of the subject, local damage may optionally occur in one or more of the same regions of the subject as the primary injury or disease, and remote tissue damage occurs in one or more different regions of the subject than the primary injury or disease. In particular embodiments, the primary injury or disease occurs in certain tissues and/or organs of the subject, and the remote or secondary injury, e.g., tissue damage, occurs in one or more different tissues and/or organs of the subject. In particular embodiments, the injured secondary tissue is muscle tissue, e.g., skeletal muscle tissue, cardiac muscle tissue, or smooth muscle tissue. In certain embodiments, the injured secondary tissue is skeletal tissue within one or more limbs (e.g., arms or legs), the diaphragm, or torso, of the subject. In certain embodiments, the injured secondary tissue is cardiac tissue of the subject. In certain embodiments, the secondary tissue damage resulting from a primary injury or disease occurs in the diaphragm or intercostal muscle. In particular embodiments, the halogen compound is iodide, e.g., NaI.

In certain embodiments, a composition of the present invention is used to treat, reduce the severity of, or prevent damage to muscle tissue resulting from a primary injury or disease in a subject in need thereof, comprising providing to said subject a pharmaceutical composition comprising a halogen compound and a pharmaceutically acceptable carrier, diluent, or excipient. In particular embodiments, the halogen compound is iodide, e.g., NaI. In particular embodiments, the muscle tissue in which damage is treated, reduced, or prevented is located within a different location or region of the subject's body than the location or region of the primary injury or disease. This may be referred to herein as a “distant” or “distal” or “remote” location or region of the subject. Cardiac muscle, the skeletal muscle regions indicated herein, and the smooth muscle regions indicated herein, all constitute different regions of the subject's body. In certain embodiments, the injured secondary tissue is skeletal tissue within one or more limbs (e.g., arms or legs), the diaphragm, or torso, of the subject. In certain embodiments, the secondary tissue damage resulting from a primary injury or disease occurs in the diaphragm or intercostal muscle. Thus, in particular embodiments, a composition disclosed herein reduces the severity of tissue damage resulting from a remote distant injury or disease in a subject, e.g., a primary injury or disease. In certain embodiments, the tissue damage results in tissue weakness.

In certain embodiments, the muscle tissue in which tissue damage is treated, reduced, or prevented is skeletal muscle tissue, cardiac muscle tissue, or smooth muscle tissue. In particular embodiments, the tissue damage is an ischemia reperfusion injury. In certain embodiments, the muscle tissue is limb muscle tissue, respiratory muscle tissue or cardiac muscle tissue. Acute lung injury that clinically manifests as acute respiratory distress syndrome (ARDS) may be treated by the methods disclosed herein. In certain embodiments, the secondary tissue damage occurs in the diaphragm or intercostal muscle, and may result in respiratory disease or difficulty in breathing.

Skeletal muscle is a form of striated muscle, typically attached to a bone. There are at least 656 skeletal muscles in the human body. In some embodiments, the skeletal muscle is located in the following region of the subject, including but not limited to any of the following: head, e.g., forehead/eyelid, extraocular muscles, ear, nose, mouth, mastication, tongue (e.g., extrinsic muscle or intrinsic muscle), soft palate, pharynx, larynx; neck, e.g., clavicular, suprahyoid, infrahyoid/strap; neck (e.g., anterior, lateral, or posterior); torso, e.g., back, chest, abdomen, pelvis, perineum; upper limbs, e.g., vertebral column, thoracic walls, shoulder, arm (e.g., anterior compartment or posterior compartment), forearm (e.g., anterior compartment, either superficial or deep or posterior compartment, either superficial or deep); hand, e.g., lateral volar (e.g., thenar, medial volar, or intermediate); lower limb, e.g., iliac region, gluteal, thigh (e.g., anterior compartment, posterior compartment/hamstring, medial compartment), leg (e.g., anterior compartment or posterior compartment, either superficial or deep), lateral compartment; or foot, e.g., dorsal, plantar (e.g., first layer, second layer, third layer, or fourth layer). In certain embodiments, the skeletal muscle is intercostal muscle.

Cardiac muscle is an involuntary, striated muscle that constitutes the main tissue of the walls of the heart.

Smooth muscle generally forms the supporting tissue of blood vessels and hollow internal organs, including but not limited to regions of the subject's body including the lungs, stomach, intestine, and bladder.

In particular embodiments of the disclosed methods, the tissue damage that is treated, reduced, or prevented, results from inflammation.

In particular embodiments, the tissue damage that is treated, reduced, or prevented results from inflammation, sepsis, or SIRS, e.g., inflammation, sepsis or SIRS resulting from the primary injury or disease in the subject. In certain embodiments, the tissue damage is distant (or distal) tissue or organ damage and even multiple organ dysfunction syndrome (MODS). Acute lung injury that clinically manifests as acute respiratory distress syndrome (ARDS) is a major component of MODS of various etiologies. Patients with an attack of ARDS who survive the initial inflammatory insult may die following a relatively minor second event that would not normally be life-threatening. According to the two-hit hypothesis, the initial overactive SIRS primes the inflammatory response. Recovery is possible if no further insult occurs. However, a relatively minor secondary event such as a line or chest infection will lead to an exaggerated secondary inflammatory response and possibly death. Inflammatory mediators play a key role in the pathogenesis of ARDS, which is the primary cause of death in these conditions. When SIRS leads to MODS and organ failure, the mortality becomes high and can be more than 50%.

A primary injury or disease in a subject may be any of a wide variety of injuries, diseases, disorders, or infections.

In some embodiments, the primary injury or disease may be an acute episode or exasperation of a chronic disease. For example, the acute episode may result in new or increased remote tissue damage, as compared to the chronic disease. In some embodiments, the chronic disease is chronic obstructive pulmonary disease (COPD), chronic heart failure, kidney disease, liver disease, pancreatitis, gastritis, cancer, or infection.

In some instances, the primary injury or disease is a localized trauma. A localized trauma refers to trauma that occurs at one or more regions of a subject's body, but not the entire body. Non-limiting examples of localized traumas include blunt force trauma, gunshot injury, stabbing injury, a surgery, e.g., cardiopulmonary bypass, a burn injury, an ischemic injury, an ischemia reperfusion injury, a traumatic brain injury, a stroke, fractures or multiple fractures, air or amniotic fluid emboli, or a radiation injury.

In some embodiments, the primary injury or disease is a medical treatment. For example, the secondary injury could result from treatment with drugs, e.g., chemotherapeutic agents, immunotherapy, or surgery. In one embodiments, the primary injury is CAR T-cell therapy to treat a tumor in the subject, which results in secondary injury or secondary tissue damage.

In some instances, the primary injury or disease is an infection, optionally a viral infection, a fungal (e.g., yeast) infection, or a bacterial infection. The infection may be local, i.e., present within one or more regions of the subject's body, or it may be systemic, i.e., in the subject's bloodstream.

In some instances, the primary injury or disease is an inflammatory condition, such as, but not limited to, local inflammatory conditions such as gastritis, pancreatitis, necrotizing enterocoloitis, or colitis. Inflammatory conditions leading to tissue or organ damage, dysfunction and failure is a major problem after injury in many other clinical conditions, such as, e.g., sepsis, shock, severe burns, acute pancreatitis, haemorrhagic shock, severe extrathoracic trauma, drug overdose, multiple transfusions, eclampsia, disseminated intravascular coagulation, and trauma.

In certain embodiments, the primary injury or disease provokes a systemic immune or inflammatory response in the subject. In some instances the primary injury or disease is an autoimmune disease. In some instances, the primary injury or disease results in a systemic inflammatory response syndrome (SIRS) or sepsis in the subject. Sepsis is defined as a SIRS in which there is an identifiable focus of infection. In particular embodiments, the tissue damage that is treated, reduced, or prevented is caused by an autoimmune disease, an inflammatory response, such as SIRs, or sepsis, that results in the tissue damage.

In general terms, systemic inflammatory response syndrome (SIRS) is an entirely normal response to injury. Systemic leukocyte activation, however, is a direct consequence of a SIRS and if excessive, can lead to distant tissue or organ damage and even multiple organ dysfunction syndrome (MODS). When SIRS leads to MODS and organ failure, the mortality becomes high and can be more than 50%. Acute lung injury that clinically manifests as acute respiratory distress syndrome (ARDS) is a major component of MODS of various etiologies.

Several infective and non-infective causes of SIRS are recognized, and any of these may be the primary injury or disease. Infective causes of SIRS include sepsis and septic shock, infection caused by bacterial pathogens, viruses, fungi, and parasites. Non-infective causes of SIRS include but are not limited to haemorrhagic shock, acute pancreatitis, and burns. Systemic leukocyte activation (cytokine-mediated) is a direct consequence of a SIRS and if excessive, can lead to MODS and multiple organ failure. In an overactive SIRS response, leukocytes become activated within the general circulation and lodge within the pulmonary microcirculation. As the condition develops, leukocytes migrate into the pulmonary interstitium and increased endothelial permeability leads to tissue edema. Leukocytes in the lungs both respond and contribute to the inflammatory process in ARDS.

In particular embodiments, methods disclosed herein are used to treat, reduce the likelihood or severity of SIRS, sepsis, or MODs following a primary clinical condition, e.g., an injury or disease.

In particular embodiments, the disclosure includes a method of treating, inhibiting, reducing the severity of, or preventing a secondary muscle tissue injury in a subject in need thereof, comprising providing to the subject an effective amount of iodide, e.g., NaI, wherein the secondary muscle tissue injury results from a primary injury or disease selected from: sepsis, COPD, chronic or acute heart failure, uremia, kidney disease, liver disease, chemotherapy, immunotherapy, pancreatitis, gastritis, and viral infection (e.g., cytomegalovirus (CMV) infection), wherein at least part (or all) of the secondary tissue damage occurs at a anatomically distance site than the primary injury or disease. In certain embodiments, the treatment results in reduced muscle weakness or increased muscle strength as compared to in the absence of treatment with the iodide. In some embodiments, the subject is provided with the iodide in one or more doses, wherein multiple doses may be provided over a period of time, e.g., a day, a week, etc. In some embodiments, each dose comprises about 1 mg/kg or about 2 mg/kg of sodium iodide.

Accordingly, the methods disclosed herein may be used to enhance the survivability of the subject following the primary injury or disease (or PICS), or following the development of SIRS, sepsis, or MODS in the subject. Enhancing survivability means increasing the likelihood that the subject will survive and not die following the primary clinical condition, or following the development of SIRS, sepsis, or MODS. Methods disclosed herein may be practiced to reduce circulating creatine kinase or cardiac troponin levels in a subject, e.g., following an ischemic injury or an ischemia reperfusion injury in the subject. In particular embodiments, the concentration of creatine kinase or cardiac troponin present in the subject blood or plasma is reduced by at least 10%, at least 20%, at least 30%, at least 40%, or at least 50%, as compared to the concentration present at some time following the injury but before treatment. In particular embodiments, the method comprises providing iodide, e.g., NaI to the subject, e.g., orally or parenterally.

Methods disclosed herein may be practiced to reduce edema in a tissue or organ in a subject, e.g., following an ischemic injury or an ischemia reperfusion injury in the subject. In particular embodiments, the edema is reduced by at least 10%, at least 20%, at least 30%, at least 40%, or at least 50%, as compared to the concentration present at some time following the injury but before treatment. In certain embodiments, the edema is present in a muscle or lung, and in certain embodiments, the edema is present in a muscle tissue, e.g., cardiac muscle, skeletal muscle or smooth muscle, such as lung muscle tissue. In particular embodiments, the method comprises providing iodide, e.g., NaI to the subject, e.g., orally or parenterally.

PICS

Post-intensive care syndrome (PICS) describes a collection of health problems that remains with patients after surviving critical illness and intensive care beyond discharge. The symptoms of PICS include new or worsening impairments in cognition, psychological health, and physical function. A patient with PICS may exhibit just one, a combination, or all three of the symptoms.

Cognitive impairment includes, but is not necessarily limited to, deficits in executive function, memory, attention, mental processing speed, and problem solving. It is a major risk factor for survivors of critical illness, and is associated with the duration of intensive care unit (ICU) delirium, acute brain dysfunction, hypotension, glucose dysregulation, respiratory failure requiring prolonged mechanical ventilation, severe sepsis, use of renal replacement therapy, acute respiratory distress syndrome (ARDS), and prior cognitive impairment.

Psychological impairment includes, but is not necessarily limited to, psychiatric illness in the form of depression, anxiety, or post-traumatic stress disorder. It is a major risk factor for survivors, and is associated with severe sepsis, acute respiratory distress syndrome, respiratory failure, trauma, hypoglycemia, and hypoxemia. Patients often develop problems with falling or staying asleep and may have nightmares and unwanted memories.

Physical impairment includes, but is not necessarily limited to, frailty, muscle wasting and weakness, poor mobility, recurrent falls, and quadri paresis or tetra paresis. Intensive care unit-acquired weakness (ICUAW) refers collectively to a set of neuromuscular syndromes commonly associated with the muscle weakness and paralysis in survivors of critical illness. These neuromuscular syndromes include critical illness polyneuropathy (CIP), critical illness myopathy (CIM), and their combination (sometimes referred to as critical illness polyneuromyopathy (CIPM)). The development of ICUAW is associated with illness severity, the duration of ICU stay, prolonged mechanical ventilation (>7 days), old age, systemic inflammatory response syndrome (SIRS), sepsis, hyperglycemia and insulin resistance, corticosteroid treatment, treatment with neuromuscular blocking agents (NMBAs), multisystem organ failure, and prolonged immobilization and sedation.

In certain embodiments, a halogen compound (e.g., iodide) or composition of the present invention is used to treat or prevent a PICS or related condition in a subject in need thereof. In particular embodiments, a composition of the present invention reduces the severity of or reduces the duration of a PICS or related condition in a subject. In particular embodiments, the subject is a mammal, e.g., a human. In particular embodiments, the composition is a stable formulation formulated to maintain the halogen compound, e.g., a halide, such as iodide or NaI, in a reduced state. In particular embodiments of any of the methods described herein, the subject is treated prior to, during, and/or following a medical treatment or intensive care. In certain embodiments, the subject is treated prior to, during, and/or after a scheduled medical treatment- or intensive care. In particular embodiments of any of the methods described herein, the PICS subject survived a medical treatment, critical illness, or intensive care. In certain embodiments, the PICS subject is discharged from a hospital. In certain embodiments, the subject has one or more PICS-associated impairment selected from physical impairment, cognitive impairment, and psychological impairment, including but not limited to any of the specific impairments disclosed herein. In particular embodiments, the subject has been diagnosed with or is considered at risk for PICS. In particular embodiments, the subject has been diagnosed with or is considered at risk for a PICS, e.g. physical impairment, cognitive impairment, or psychological impairment.

In certain embodiments, the PICS or any of the related disorders or symptoms disclosed herein occur or would occur following a scheduled or emergency medical treatment. In particular embodiments, the treatment comprises the use of anaesthesia and/or the patient is unconscious during the treatment. In particular embodiments, the medical treatment involves treating a trauma, a fall, a broken bone, a heart attack, or a stroke. In some embodiments, the trauma affects large bones, abdominal organs, chest organs, or the head. In some embodiments, the medical treatment is a surgery. In some embodiments, the surgery is abdominal, thoracic, orthopedic, cardiac, brain, lung, eye, or head and neck surgery.

In certain embodiments, PICS is triggered by sepsis, mechanical ventilation, muscle unloading, immobilization, infusion, steroid treatment, or denervation. In some embodiments, the individual has been immobilized for at least 48 hours. In some embodiments, the infusion is associated with hyperchloremic acidosis. In particular embodiments, PICS is associated with the presence and persistence of sepsis, systemic inflammatory response syndrome (SIRS), acute respiratory distress syndrome (ARDS), or multiple organ failure dysfunctions. In some embodiments, the PICS is associated with metabolic acidosis, e.g., metabolic acidosis associated with kidney dysfunction or failure. In some embodiments, the PICS is associated with diabetic acidosis, hyperchloremic acidosis, lactic acidosis, or renal tubular acidosis. In particular embodiments, PICS is related to critical illness, such as pneumonia, drug-induced organ failure, thermal injury, or peritonitis.

In some embodiments, the PICS results from treatment with drugs, e.g., chemotherapeutic agents, immunotherapy, or surgery. In one embodiments, the immunotherapy is CAR T-cell therapy, e.g., to treat a tumor in the subject, which results in PICS.

In particular embodiments, the subject being treated may be considered more vulnerable to a PICS, e.g., the subject may be 50 or older, 60 or older, 65 or older, 70 or older, 75 or older, or 80 or older. In particular embodiments, treatment with one or more medications during intensive care makes the subject more vulnerable to PICS, e.g. corticosteroids or neuromuscular blocking agents. In certain embodiments, the subject is provided an effective amount of a halide, e.g., iodide, such as NaI, that decreases the likelihood that the PICS or related disorder occurs in the subject, reduces the severity of one or more symptoms of the PICS or related disorder in the subject, or reduces the duration of one or more symptoms of the PICS or related disorder in the subject. In particular embodiments, the halide is provided to the subject parenterally, e.g., intravenously or by infusion, e.g., a bolus infusion, prior to the medical treatment and/or during the medical treatment.

In particular embodiments, PICS symptoms include or manifest as myopenia or ICUAW. The term myopenia has been suggested to describe physical weakness associated with the loss of muscle mass and/or strength. Also referred to as muscle wasting or muscle wasting disease, myopenia is frequently observed in a wide variety of acute and chronic diseases and conditions, and is associated with decreased quality of life and increased risk of morbidity and mortality. The recent inclusions of sarcopenia (muscle wasting associated with old age) and cachexia (muscle wasting associated with chronic illness, ie. cancer, heart failure, and kidney disease) in the international classification of diseases (ICD) reflects the growing appreciation of the clinical outcomes and health care burden associated with myopenia, and also its relevance as a therapeutic target. ICUAW, an acute myopenia, generally describes the unaccountable muscle wasting and weakness that commonly develops in critically ill patients during their stay in the ICU, and for survivors of critical illness, its diagnosis is predictive of long term muscle weakness and functional disability, and is associated with increased risk of mortality and poor health-related quality of life. ICUAW refers collectively to a set of neuromuscular syndromes, critical illness polyneuropathy (CIP), critical illness myopathy (CIM), and critical illness polyneuromyopathy (CIPM or CINM), distinguished primarily by whether weakness involves the nerves supplying the muscle (CIP), the muscle tissue only (CIM), or a combination of the two (CIPM). CIP describes an impaired neuronal excitation and stimulation of muscle contraction characterized by sensorimotor axonal degeneration and denervation of muscle tissue. CIM refers to muscle weakness in the absence of neuropathy, and is characterized by atrophy of type II muscle fibers and preferential myosin protein loss.

The pathophysiology of neuromuscular damage and dysfunction in ICUAW is complex and multifactorial. Increased vascular permeability associated with microcirculatory dysfunction is common in critically ill patients and is thought to promote delivery of cytotoxic substances to nerve fibers. Edema, also a consequence of increased permeability, can impair oxygen delivery, and thus cellular energy production, in neurons and myocytes. Insulin resistance and hepatic gluconeogenesis contribute to hyperglycemia, all of which are common features of critical illness. High levels of glucose have direct cytotoxic effects on neuromuscular tissue, and also contribute to mitochondrial dysfunction.

Certain cytokines, including TNF-α, IL-1, and IL-6, are elevated in critically ill patients and have a number of direct effects on myocytes, culminating in muscle loss and a net catabolic effect on muscle cell protein. These effects include death receptor-mediated induction of myocyte apoptosis, enhancement of the proteolytic activity of calpain and ubiquitin proteases, and inhibition of PI3K/Akt-mediated anabolic signaling. TNF-α and IL-1 can also depress the contractile force of skeletal muscle.

In various embodiments methods disclosed herein are used to reduce the level of one or more cytokine in a subject. In particular embodiments, the cytokine is IL-6, IL-10, KC, or MIP-2. In particular embodiments, one or more of IL-6, IL-10, KC, or MIP-2 is significantly reduced in muscle tissue or plasma. In one embodiments, IL-6 is significantly reduced in plasma. In certain embodiments, one or more cytokine is reduced by at least 10%, at least 20%, at least 30%, at least 40%, or at least 50%, e.g., in either muscle tissue or plasma. Thus, the methods disclosed herein may be used to reduce intramuscular and/or systemic inflammation in a subject.

In various embodiments of any of the methods disclosed herein, the severity of the one or more symptoms or the duration of the one or more symptoms is reduced by at least 10%, at least 20%, at least 30%, at least 40%, at least 50%, at least 60%, at least 70%, at least 80%, or at least 90%.

In certain embodiments, the present invention includes methods of treating or preventing PICS in a subject in need thereof, comprising providing to the subject an effective amount of a halide or a composition of the present invention. In particular embodiments, the composition comprises an effective amount of one or more halides and optionally one or more additional active agents, including any of those described herein. In particular embodiments, the halide or halogen compound comprises iodine, e.g., iodide or iodate, such as NaI. In particular embodiments, the composition also comprises glutathione or another reducing agent.

In some embodiments, the present invention includes methods of treating or preventing a symptom associated with PICS in a subject in need thereof, comprising providing to the subject an effective amount of a composition of the present invention. In particular embodiments, the composition comprises iodide, e.g., NaI.

In particular embodiments, compositions of the present invention are used to treat subjects who have been diagnosed with or who are susceptible to PICS, e.g., a subject scheduled to undergo a medical treatment or intensive care that may cause PICS. In certain embodiments, the subject is treated prior to, during, and/or following the medical treatment or intensive care. In some embodiments, the subject is treated with a bolus of halide, e.g., iodide, such as NaI, prior to the medical treatment or intensive care. The subject may also be treated with halide, e.g., NaI during and/or following the medical treatment or intensive care. In some embodiments, the methods are used to prevent or reduce the likelihood of PICS occurring in a subject, to prevent or inhibit the occurrence of PICS in the subject, or to prevent or reduce the severity of PICS in the subject. In particular embodiments, methods of the present invention are used to treat, inhibit or prevent any of the disorders or symptoms associated with PICS in a subject.

In one embodiment, the disclosure provides a method of treating, preventing, or reducing the severity or duration of a PICS or related disorder in a subject in need thereof, comprising providing to the subject an effective amount of a halide, e.g., iodide, such as NaI, before and/or during a portion of time while the subject undergoes a medical treatment or intensive care procedure.

In some embodiments, methods disclosed herein are used to treat PICS, including but not limited to an associated physical impairment, cognitive impairment, or psychiatric impairment, in a subject who survived a critical illness and intensive care and is beyond discharge.

In certain embodiments, methods and compositions disclosed herein are used to treat or prevent a PICS-associated cognitive impairment in a subject in need thereof. Such cognitive impairments include, but are not limited to, deficits in executive function, memory, attention, mental processing speed, and problem solving.

In certain embodiments, methods and compositions disclosed herein are used to treat or prevent a PICS-associated psychological impairment in a subject in need thereof. Such psychological impairments include, but are not limited to, psychiatric illness in the form of depression, anxiety, or post-traumatic stress disorder.

In certain embodiments, methods and compositions disclosed herein are used to treat or prevent a PICS-associated physical impairment in a subject in need thereof. Such physical impairments include, but are not limited to, intensive care unit (ICU)-acquired neuromuscular weakness in form of critical illness polymeuropathy (CIP), critical illness myopathy (CIM), sepsis-induced myopathy (SIM), steroid-denervation myopathy (SDM), prolonged neuromuscular blockade, disuse atrophy, poor mobility, recurrent falls, quadri paresis or tetra paresis.

Dosing and Administration

In certain embodiments, the halogen compound (e.g., iodide, such as NaI) is provided to the subject in a liquid pharmaceutical composition comprising a pharmaceutically acceptable carrier, diluent, or excipient. In some embodiments, at least 90% of the halogen compound in the composition is present in a reduced form for at least one hour, at least one week, at least one month, or at least six months when stored at room temperature. In particular embodiments, a composition comprising the halogen compound comprises one or more of a reducing agent, a tonicity agent, a stabilizer, a surfactant, a lycoprotectant, a polyol, an antioxidant, or a preservative.

In particular embodiments, the pharmaceutical composition is provided to the subject prior to, during, or following the primary injury or disease, or the medical procedure.

In certain embodiments, the pharmaceutical composition is provided to the subject orally or parenterally. For example the pharmaceutical composition may be provided to the subject as a bolus dose prior to the primary injury or disease, or medical treatment, optionally wherein the bolus dose comprises less than or equal to about 10 mg/kg of halogen compound (e.g., NaI), optionally about 1.0 mg/kg or about 2 mg/kg. In other examples, the pharmaceutical composition is provided to the subject following the primary injury or disease or medical treatment. In some embodiment, multiple doses of the halogen compound (e.g., NaI) are provided to the subject. In particular embodiments, each dose comprises less than or equal to about 10 mg/kg of the halogen compound, optionally about 1.0 mg/kg or about 2.0 mg/kg of the halogen compound (e.g., NaI). In certain embodiments, multiple doses of the halogen compound are provided to the subject over a period of time, e.g., 4 hours, 8 hours, 12 hours, 1 day, 2 days, four days, 1 week, 2 weeks, 3 weeks, 1 month, 2 months, 4 months, 8 months, 1 year, or longer. In certain embodiments, the halogen compound (e.g., NaI) is provided to the subject as a continuous infusion, optionally prior to and/or during and/or following the primary injury or disease or medical treatment. In certain embodiments, less than about 100 mg/kg of iodide is provided to the subject over a period of time, e.g., 4 hours, 8 hours, 12 hours, 1 day, 2 days, four days, 1 week, 2 weeks, 3 weeks, 1 month, 2 months, 4 months, 8 months, 1 year, or longer.

In particular embodiments of any of the methods disclosed herein, the halogen compound is an iodide, e.g., sodium iodide (NI).

In particular embodiments of any of the methods disclosed herein, the subject is a mammal, e.g., a human.

In particular embodiments, the composition is a stable formulation formulated to maintain the halogen compound, e.g., a halide, such as iodide or NaI, in a reduced state.

In certain embodiments of methods of the invention, the halogen compound comprises iodine or iodide, e.g., NaI, and the effective amount is greater than or equal to about 150 μg, greater than or equal to about 300 μg, greater than or equal to about 500 μg, greater than or equal to about 1 mg, greater than or equal to about 2 mg, greater than or equal to about 5 mg, greater than or equal to about 10 mg, greater than or equal to about 15 mg, or greater than or equal to about 20 mg. In certain embodiments, the effective amount is 150 μg to 1000 mg, 300 μg to 1000 mg, 500 μg to 1000 mg, 1 mg to 1000 mg, 2 mg to 1000 mg, 5 mg to 1000 mg, 10 mg to 1000 mg, 150 μg to 100 mg, 300 μg to 100 mg, 500 μg to 100 mg, 1 mg to 100 mg, 2 mg to 100 mg, 5 mg to 100 mg, or 10 mg to 100 mg. In certain embodiments, the effective amount is 150 μg to 50 mg, 300 μg to 20 mg, 500 μg to 10 mg, 1 mg to 20 mg, 1 mg to 10 mg, or about 5 mg, about 10 mg, about 15 mg, or about 20 mg.

In particular embodiments of any of the methods of the present invention, a subject is treated with or contacted with an effective amount of a composition or compound of the present invention, wherein said effective amount of about 0.01 mg/kg to about 20 mg/kg, about 0.05 mg/kg to about 10 mg/kg, about 0.1 mg/kg to about 5 mg/kg, about 0.5 mg/kg to about 2 mg/kg, about 0.5 mg/kg to about 1 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg or about 1.2 mg/kg. In certain embodiments, the composition comprises a halogen compound.

In particular embodiments, an effective amount of iodine or iodide is an amount at least or about two-fold, three-fold, four-fold, five-fold, six-fold, seven-fold, eight-fold, nine-fold, ten-fold, twelve-fold, fifteen-fold, twenty-fold, fifty-fold, 100-fold, 1,000-fold, 10,000-fold or 100,000-fold of the average daily recommended amounts as listed below. In particular embodiments, the effective amount of iodine or iodide is an amount between two-fold and twenty-fold, between five-fold and fifteen-fold, or between five-fold and ten-fold of the average daily recommended amounts of iodine as listed below.

| Life Stage | Recommended Amount¹ (mcg) |
|---|---|
| Birth to 6 months | 110 |
| Infants 7-12 months | 130 |
| Children 1-8 years | 90 |
| Children 9-13 years | 120 |
| Teens 14-18 years | 150 |
| Adults | 150 |
| Pregnant teens and women | 220 |
| Breastfeeding teens and women | 290 |

¹NIH Office of Dietary Supplements Iodine Fact Sheet for Consumers, reviewed Jun. 24, 2011, obtained 2013.

In certain embodiments of methods of the invention, the halogen compound comprises iodine, e.g., NaI, and the effective amount is about 0.01 mg/kg to about 20 mg/kg, about 0.05 mg/kg to about 10 mg/kg, about 0.1 mg/kg to about 5 mg/kg, about 0.5 mg/kg to about 2 mg/kg, about 0.5 mg/kg to about 1 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg or about 1.2 mg/kg. In certain embodiments, the halogen compound comprises iodine, and the effective amount is an amount that achieves about the same concentration or amount that is achieved by an effective amount of iodine that is at least or about two-fold, three-fold, four-fold, five-fold, six-fold, seven-fold, eight-fold, nine-fold, ten-fold, twelve-fold, fifteen-fold, or twenty-fold of the average daily recommended amounts as listed below.

In certain embodiments of methods disclosed herein, a subject in need thereof is provided with an effective amount of a halogen compound, e.g., a halogen compound comprising iodine, bromine, or fluorine, an iodide, such as e.g., sodium iodide, potassium iodide, magnesium iodide, hydrogen iodide, calcium iodide, or silver iodide. In particular embodiments, the halogen compound is provided parenterally, orally or systemically in an amount sufficient to achieve a blood concentration of 20 parts per billion (ppb) to 20 parts per million (ppm). In particular embodiments, the subject is a human.

In certain embodiments, the composition is provided to the subject in an amount sufficient to increase the blood concentration of the halogen compound at least five-fold, at least ten-fold, at least 50-fold, at least 100-fold, at least 500-fold, or at least 1000-fold for at least some time.

A composition comprising the halogen compound, and a composition comprising an additional active agent may be provided to the subject at the same time, at different times, or during overlapping time periods. In particular embodiments when both are present, the halogen compound and the additional active agent are present in the same or different compositions.

In particular embodiments where the composition comprises glutathione and a halogen compound, glutathione is present in an amount sufficient to inhibit oxidation of the halogen compound, including any of the ranges described herein. In particular embodiments, the halogen compound is iodide, e.g., NaI.

In various embodiments of methods of the present invention, a subject is exposed to a composition of the current invention for about, at least, at least about, or at most about 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4 weeks, 1, 2, 3, 4, 5, 6, 7, 8 or 9 months or more, and any range or combination therein.

Furthermore, when administration of a composition according to the present invention is intravenous or by infusion, it is contemplated that the following parameters may be applied. A flow rate of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or gtts/min, or any range derivable therein. In some embodiments, the amount of the composition is specified by volume, depending on the concentration of the halogen compound in the composition. An amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

Volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or liters, or any range therein, may be administered overall or in a single session.

According to various embodiments of the methods of the present invention, a subject is provided with a composition of the invention, e.g., intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, intraocularly, subcutaneously, subconjunctival, intravesicularly, mucosally, intrapericardially, intraumbilically, intraocularally, orally, topically, locally, by injection, by infusion, by continuous infusion, by absorption, by adsorption, by immersion, by localized perfusion, via a catheter, or via a lavage. In particular embodiments, it is provided parenterally, e.g., intravenously, or by inhalation. “Parenteral” refers to any route of administration of a substance other than via the digestive tract. In specific embodiments, a halogen compound is provided to the subject by intravenous administration or infusion.

In additional embodiments, methods of the present invention include a drug delivery device designed to limit, prevent or inhibit oxidation of a reduced form of an active agent, such as, e.g., a reduced form of a halogen compound, such as NaI. In specific embodiments, the device maintains a reduced form of an active agent in its reduced form. In particular embodiments, the device comprises the reduced form of an active agent, such as the reduced form of a halogen compound, for example. In specific embodiments, the drug device comprises a composition of the present invention.

Manufacturer-prepared, premixed ready-to-use products represent a useful approach to intravenous drug safety, since they remove error associated with measuring and diluting intravenous or infused drugs. Accordingly, in certain embodiments, the present invention includes a drug delivery device for administration of a ready-to-use product comprising a reduced form of an active agent. In particular embodiments, the reduced form of active agent is a reduced form of a halogen compound, e.g., NaI.

In related embodiments, the present invention comprises a container having therein an effective amount of a composition of the present invention or an effective amount of a halogen compound. The effective amount may be in liquid form, e.g., the active agent may be dissolved in a solution, or it may be in dry form (e.g., dried, lyophilized, or freeze-dried), such that the active agent may be dissolved in a solution prior to administration to a subject.

In all embodiments of compositions described herein, it is understood that the composition may be a pharmaceutical composition.

### EXAMPLES

### Example 1

Iodide Protects Skeletal Muscle from Damage Following Hind Limb Ischemia-Reperfusion Injury

Anesthetized adult male C57BL/6 mice were subjected to hindlimb ischemia reperfusion injury (bilateral leg tourniquets for 2 hours (ischemia) followed by 3 hours of reperfusion after tourniquet removal). To determine whether exogenous iodide could reduce damage to heart and lung tissue caused by the ischemia reperfusion injury, various doses of iodide (0 mg/kg, 1 mg/kg, 10 mg/kg or 20 mg/kg) were administered intravenously (i.v.) by the retro orbital (r.o.) to the animals 5 minutes prior to reperfusion of the hind limb.

The animals were then sacrificed and blood (plasma) and lung and GC muscle samples obtained. The blood (plasma) samples were assayed to determine the concentrations of creatine kinase and cardiac troponin present in each. In addition, the amount of edema present in the lung and GC muscle was determined as wet weight/dry weight.

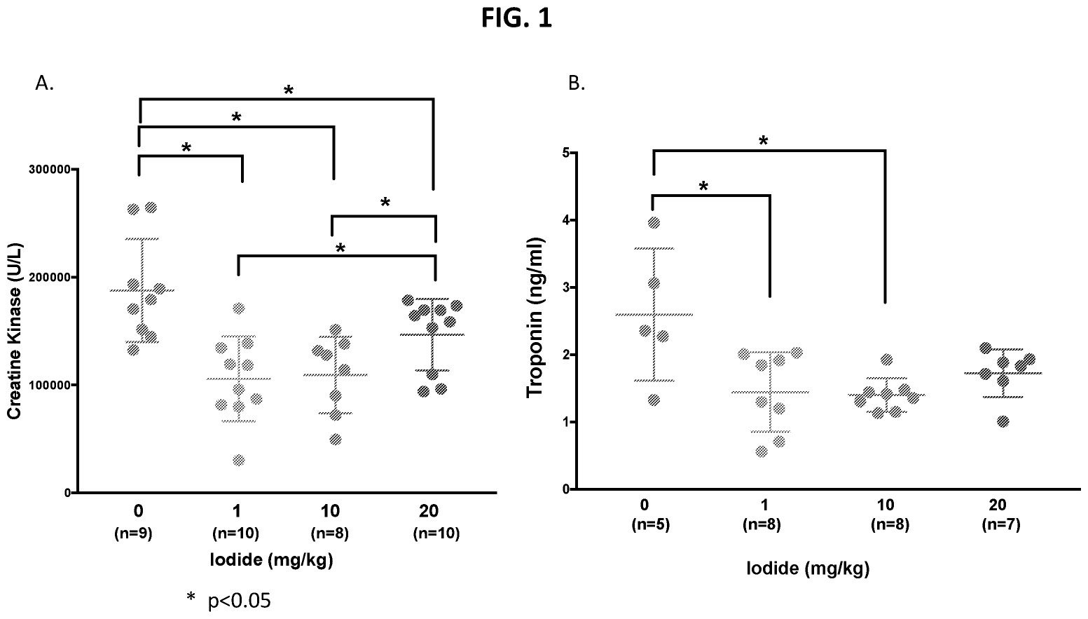

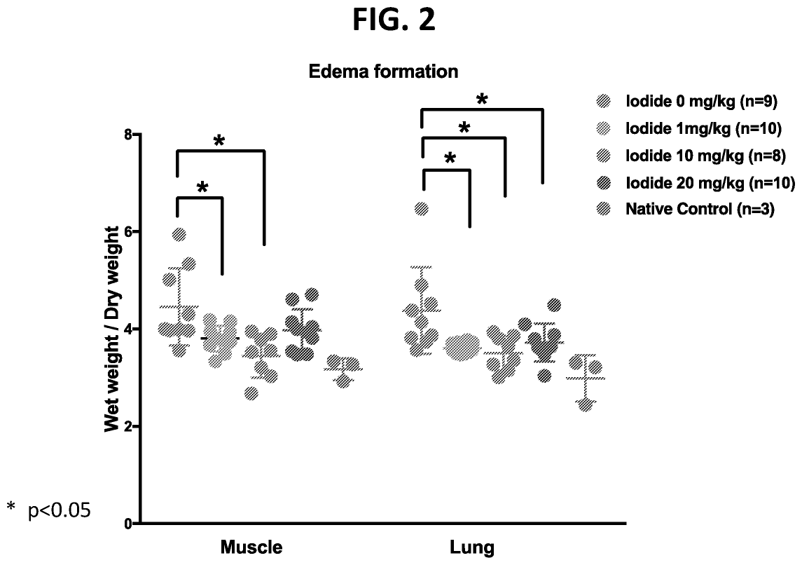

As shown in FIG. 1A and FIG. 1B, levels of creatine kinase (CK) and cardiac troponin were significantly reduced in animals treated with iodide. Administration of 1 mg/kg, 10 mg/kg, or 20 mg/kg sodium iodide significantly reduced the plasma CK levels compared to vehicle administration (FIG. 1A). The level of cardiac troponin was significantly reduced following i.v. administration of 10 mg/kg (FIG. 1). In addition, both pulmonary edema (lung) and muscle edema (gastrocnemius) were reduced following i.v. administration of iodide (FIG. 2).

Since circulating levels of creatine kinase and cardiac troponin are indicative of muscle damage, these results indicate that treatment with iodide reduces or prevents skeletal and cardiac muscle damage that occurs at sites distant from the hind limb iscemia reperfusion injury. In addition, treatment with iodide resulted in reduced edema in both muscle and lung following hind limb ischemia reperfusion injury, further demonstrating the treatment with iodide protects tissues distant from the location of ischemia reperfusion injury from damage.

### Example 2

### Sodium Iodide is Efficacious in the Treatment of Systemic Inflammation

Studies were performed to demonstrate the efficacy of sodium iodide (NaI), delivered either intravenously (i.v.) or orally (p.o.), to reduce systemic inflammation and improve mortality.

Study A

A bilateral hind-limb ischemia model was used to induce systemic inflammation. Male C57Bl/6 mice (7-10 weeks old) were subjected to 2 hours of bilateral hind limb ischemia followed by 3 hours of reperfusion. Ischemia was induced by application of latex o-rings above the greater trochanter using a McGivney Hemorrhoidal ligator. After 3 hours of reperfusion, the plasma was analyzed for levels of creatine kinase (CK), or other markers of organ injury: blood urea nitrogen (BUN), alanine aminotransferase (ALT), and aspartate aminotransferase (AST).

A 1, 3, 10, or 20 mg/kg bolus of sodium iodide was administered intravenously (i.v.) by the retro orbital (r.o.) route 5 minutes prior to reperfusion of the hind limbs.

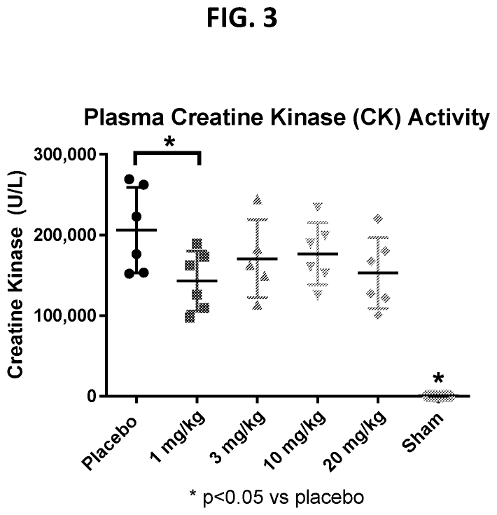

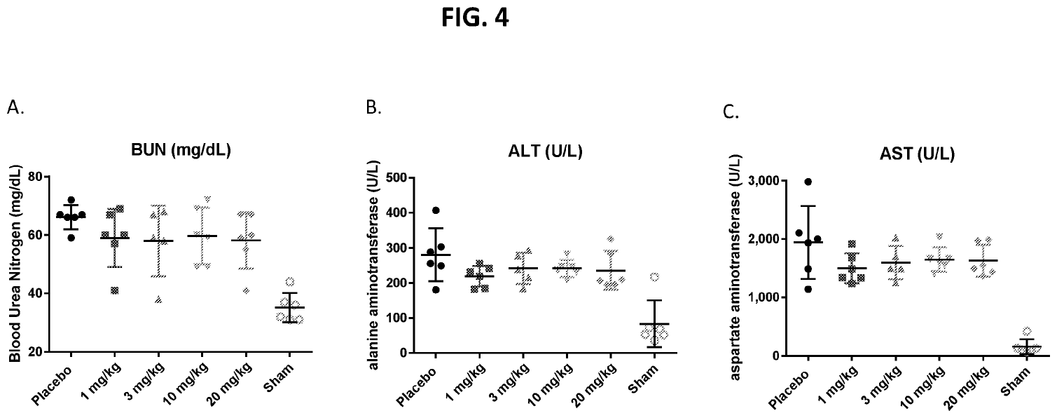

Administration of 1 mg/kg sodium iodide significantly reduced the plasma CK levels compared to vehicle administration (FIG. 3). The plasma levels of blood urea nitrogen (BUN), alanine aminotransferase (ALT), and aspartate aminotransferase (AST), were reduced following administration of 1, 3, 10, or 20 mg/kg sodium iodide (FIGS. 4A-4C).

Study B

The model used to evaluate survival was similar to that previously described for Study A; however, the time of ischemia was increased to 2.5 hours. The time from reperfusion to death was monitored for the next 24 hours.

The drinking water of the mice was supplemented with 84 μM NaI and the mice were allowed ad libitium access (p.o.) to this water for >5 days prior to hind limb ischemia.

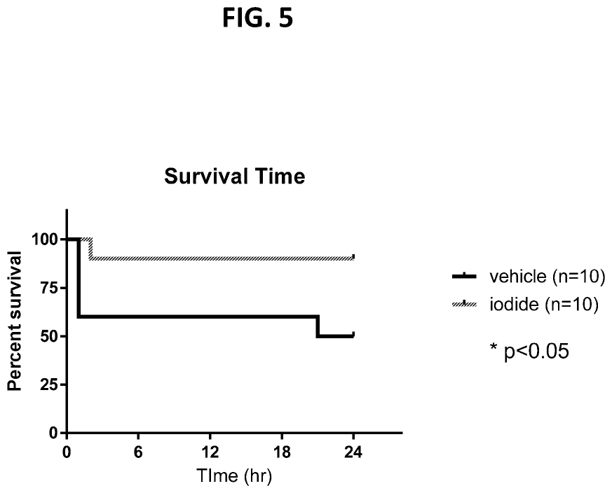

Administration of 84 μM sodium iodide significantly increased survival compared to vehicle (FIG. 5). At 24 hours, 90% of the sodium iodide treated animals were alive but only 50% of the vehicle treated animals were alive.

Study C

The model used to evaluate survival was similar to that previously described for Study A; however, the time of ischemia was increased to 3 hours. The time from reperfusion to death was monitored for the next 2 months.

A 1 mg/kg bolus of sodium iodide was administered intravenously (i.v.) by the retro orbital (r.o.) route 5 minutes prior to reperfusion of the hind limbs.

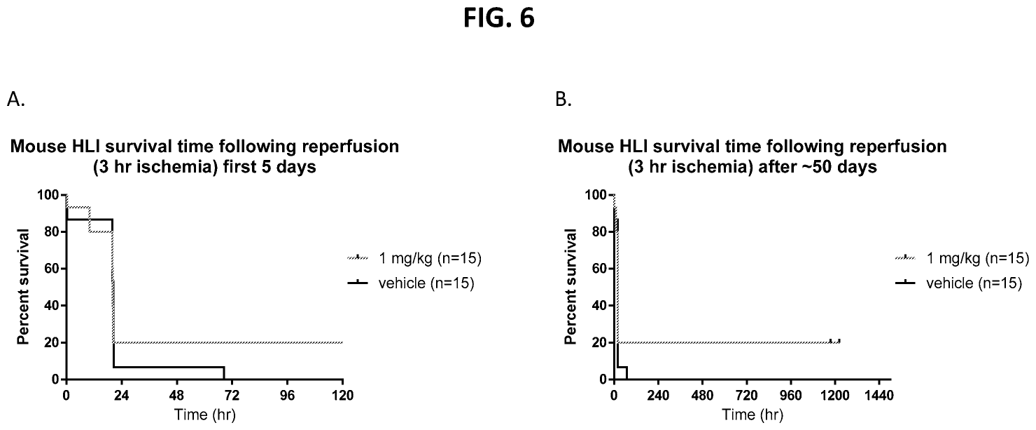

Administration of 1 mg/kg sodium iodide increased survival compared to vehicle administration. After 72 hours, 0% of the animals that received vehicle survived, however 20% of the animals that received sodium iodide were alive (FIG. 6A). These animals continued to live for another 2 months (at this point the study ended) (FIG. 6B).

### Example 3

### Sodium Iodide is Efficacious in the Treatment of Intramuscular and Systemic Inflammation

Studies were performed to demonstrate the efficacy of sodium iodide (NaI), delivered either intravenously (i.v.) or orally (p.o.), to reduce intramuscular and systemic inflammation following injury by quantitative analysis of cytokine levels in muscle and plasma.

A mouse model of hind limb ischemia was utilized to induce intramuscular and systemic inflammation. Male C56Bl/6 mice˜7-10 weeks old were subject to bilateral hind limb ischemia (HLI) by placing an O-ring on both hind limbs for 2.5 hours. Five minutes prior to reperfusion an intravenous bolus of sodium iodide (NaI; FDY-5301) at a concentration of 1 mg/kg was delivered. Vehicle treated animals received an i.v. bolus of saline. After i.v. delivery, at the 2.5 hour mark, the O-rings were cut off and removed to allow for tissue reperfusion. The mice were sacrificed 24 hours following reperfusion. Upon sacrifice, whole blood was removed (plasma was then separated and frozen at −80° C.), the gastronemius muscle was removed and snap frozen in liquid nitrogen and then stored at −80° C. The cytokines were evaluated on a MAGPIX® instrument (Luminex Corp) using a custom multiplex assay kit from MilliporeSigma (MCYTOMAG-70K-09, containing: INF-γ, IL-18, IL-2, IL-6, IL-10, KC, LIX, MIP-2 & TNF-α.

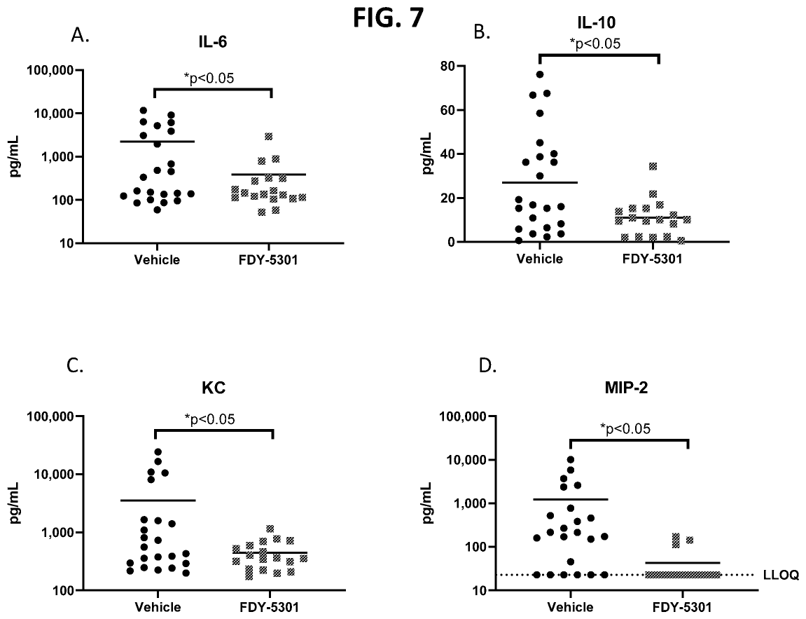

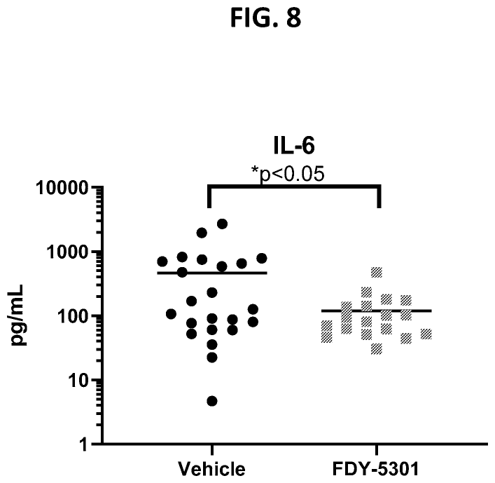

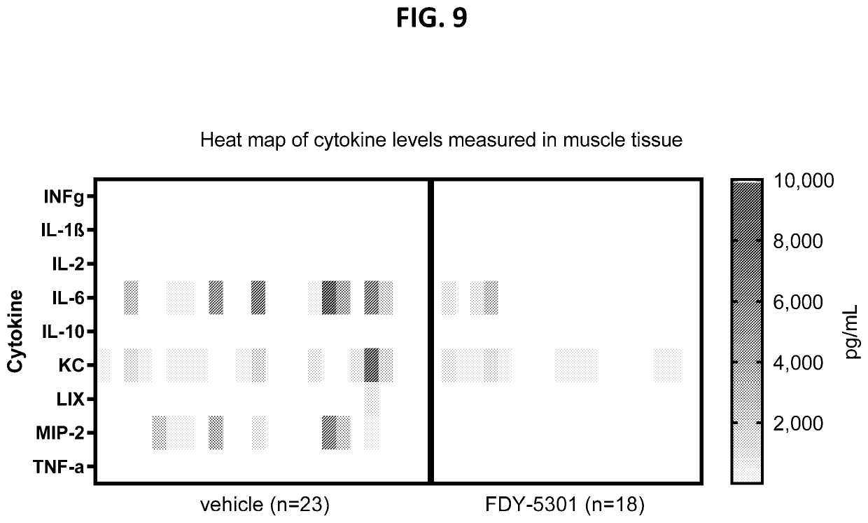

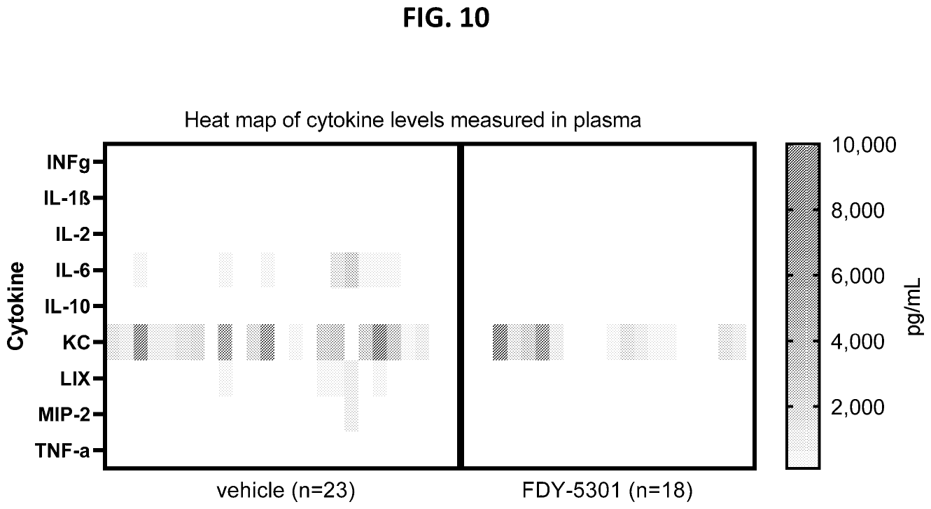

The mice treated with NaI had a significant reduction in IL-6, IL-10, KC, and MIP-2 (p<0.05) in muscle tissue (FIG. 7), and a significant reduction in plasma IL-6 (FIG. 8). Furthermore, the heat map of all cytokines assessed in muscle or plasma (FIGS. 9 and 10, respectively) indicate an overall reduction in inflammation following FDY-5301 administration. These results demonstrate that local injury can cause a systemic insult that could result in distal tissue damage.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

We claim:

1. A method for treating, reducing the severity of, or preventing a secondary injury or secondary tissue damage resulting from a primary injury or primary disease in a subject in need thereof, comprising providing to said subject an effective amount of an iodide.

2. The method of claim 1, wherein the secondary injury or tissue damage is post-intensive care syndrome (PICS).

3. The method of claim 1, wherein the secondary injury or tissue damage is tissue damage.

4. The method of claim 3, wherein said iodide is sodium iodide, potassium iodide, hydrogen iodide, calcium iodide, silver iodide, magnesium iodide, zinc iodide, or lithium iodide.

5. The method of claim 4, wherein said iodide is sodium iodide.

6. The method of any one of claims 1-5, wherein said iodide is provided to the subject in an amount sufficient to increase the blood concentration of the halogen compound at least five-fold, at least ten-fold, at least 50-fold, at least 100-fold, at least 500-fold, at least 1000-fold, at least 10,000-fold, or at least 100,000-fold for at least some time.

7. The method of any one of claims 1-6, wherein the iodide is present in a stable liquid pharmaceutical composition comprising the halogen compound and a pharmaceutically acceptable carrier, diluent, or excipient.

8. The method of claim 7, wherein at least 90% of the iodide in the composition is present in a reduced form for at least one hour, at least one week, at least one month, or at least six months when stored at room temperature.

9. The method of any one of claims 1-8, wherein said composition comprising the iodide comprises one or more of a reducing agent, a tonicity agent, a stabilizer, a surfactant, a lycoprotectant, a polyol, an antioxidant, or a preservative.

10. The method of any one of claims 1-9 wherein the iodide is provided to the subject prior to, during, or following the primary injury or disease.

11. The method of claim 10, for treating, reducing the severity of, or preventing remote tissue damage resulting from the primary injury or disease, wherein the tissue is muscle tissue, optionally cardiac muscle tissue, skeletal muscle tissue, or smooth muscle tissue.

12. The method of any one of claims 1-11, wherein the primary injury or disease is a localized trauma.

13. The method of claim 12, wherein the localized trauma is a blunt force trauma, a surgery, a burn injury, an ischemic injury, an ischemia reperfusion injury, a traumatic brain injury, a stroke, or a radiation injury.

14. The method of any one of claims 1-11, wherein the primary injury or disease is an infection, optionally a viral infection, a fungal infection, or a bacterial infection.

15. The method of any of claims 1-11, wherein the primary injury or disease is a local inflammatory condition, optionally gastritis, pancreatitis, necrotizing enterocoloitis, or colitis.

16. The method of any of claims 1-15, wherein the primary injury or disease has resulted in a systemic inflammatory response syndrome (SIRS) or sepsis in the subject.

17. The method of any one of claims 1-11, wherein the primary injury or disease is an acute episode of a chronic disease, optionally chronic obstructive pulmonary disease (COPD), heart failure (optionally left-sided, right-sided, systolic, diastolic or congestive heart failure), uremia, kidney disease, liver disease, pancreatitis, gastritis, or bacterial, viral or fungal infection.

18. The method of any of claims 1-17, wherein the primary injury or disease is present within a different region of the subject than the secondary injury or remote tissue damage, optionally within a different organ, tissue, or limb.

19. The method of any one of claims 1-10 for treating, reducing the severity of, or preventing PICS in the subject wherein the iodide is provided to the subject prior to, during, or following a medical procedure or critical care.

20. The method of claim 19, wherein the treatment inhibits onset of, reduces severity of, or improves one or more cognitive impairments in the subject, wherein the one or more cognitive impairment is optionally elected from: deficits in executive function, memory, attention, mental processing speed, and problem solving.

21. The method of claim 19, wherein the treatment inhibits the onset of, reduces severity of, or improves one or more psychological impairments in the subject, wherein the one or more psychological impairment is optionally selected from: psychiatric illness in the form of depression, anxiety, and post-traumatic stress disorder.

22. The method of claim 19, wherein the treatment inhibits the onset of, reduces severity of, or improves one or more physical impairments in the subject, wherein the one or more physical impairment is optionally selected from: intensive care unit (ICU)-acquired neuromuscular weakness, optionally in form of critical illness polyneuropathy (CIP), critical illness myopathy (CIM), prolonged neuromuscular blockade, prolonged mechanical ventilation, disuse atrophy, prolonged immobility, poor mobility, recurrent falls, quadri paresis and tetra paresis.

23. The method of claim 19, wherein the treatment treats or prevents metabolic acidosis, diabetic acidosis, hyperchloremic acidosis, lactic acidosis, or renal tubular acidosis.

24. The method of any one of claims 1-23, wherein the iodide is provided to the subject orally or parenterally.

25. The method of any one of claims 1-24, wherein the iodide is provided to the subject as a bolus dose, optionally prior to a medical procedure or critical care.

26. The method of any one of claims 1-24, wherein multiple doses of the iodide are provided to the subject.

27. The method of claim 25 or claim 26, wherein a dose comprises less than or equal to about 10 mg/kg of the iodide, optionally about 1.0 mg/kg or about 2.0 mg/kg of the iodide.

28. The method of any one of claims 1-24, wherein the iodide is provided to the subject as a continuous infusion, optionally prior to and/or during and/or following the primary injury or disease.

29. The method of claim 28, wherein less than about 100 mg/kg of iodide is provided to the subject.

30. The method of any one of claims 1-29, wherein the method enhances the survivability of the subject following the primary injury or disease.

31. The method of any one of claims 1-30, wherein the method results in a reduced level of one or more cytokine in a muscle tissue or plasm of the subject.

32. The method of claim 31, wherein the one or more cytokine includes IL-6, IL-10, KC, or MIP-2.

33. The method of claim 32, wherein one or more of IL-6, IL-10, KC, or MIP-2 is significantly reduced in a muscle tissue of the subject.

34. The method of claim 32, wherein IL-6 is significantly reduced in plasma of the subject.
